(19) United States
(12) Patent Application Publication
(10) Pub. No.: US 2009/0175862 A1
(43) Pub. Date: Jul. 9, 2009

(54) COMBINATION THERAPY EMPLOYING LYMPHOTOXIN BETA RECEPTOR BINDING MOLECULES IN COMBINATION WITH SECOND AGENTS

(75) Inventors: Erika Lorraine Silverio, Framingham, MA (US); Cindy Bottiglio, Billerica, MA (US)

Correspondence Address: LAHIVE & COCKFIELD, LLP / BIOGEN IDEC;FLOOR 30, SUITE 3000, ONE POST OFFICE SQUARE, BOSTON, MA, 02109-2127, US

(73) Assignee: Biogen Idec MA Inc., Cambridge, MA (US)

(21) Appl. No.: 12/330,269

(22) Filed: Dec. 8, 2008

Related U.S. Application Data
(63) Continuation of application No. PCT/US2007/014051, filed on Jun. 15, 2007.
(60) Provisional application No. 60/814,357, filed on Jun. 15, 2006.

(51) Int. Cl. A61K 39/395; A61P 35/00; C07K 16/22

(52) U.S. Cl. 424/133.1; 424/130.1; 424/158.1; 424/145.1; 530/389.2

(57) ABSTRACT

This invention features combination therapies that include a composition that activates lymphotoxin-beta receptor signaling in combination with one or more other biologic agents, as well as therapeutic methods.

### RELATED APPLICATIONS

This patent application is a continuation of PCT/US2007/014051, filed Jun. 15, 2007, which claims the benefit of U.S. Provisional Patent Application Ser. No. 60/814,357, entitled “Combination Therapy Employing Lymphotoxin Beta Receptor Binding Molecules in Combination With Second Agents”, filed Jun. 15, 2006. The entire contents of the above-referenced patent applications are incorporated herein by reference.

### BACKGROUND OF THE INVENTION

Cancer is one of the most prevalent health problems in the world today, affecting approximately one in five individuals in the United States. Many molecules have been identified on tumor cells as potential targets for antibody based therapy.

For example, lymphotoxin beta receptor (referred to herein as LT-β-R) is a member of the tumor necrosis factor family which has a well-described role both in the development of the immune system and in the functional maintenance of a number of cells in the immune system including follicular dendritic cells and a number of stromal cell types (Crowe et al. (1994) Science 264:707; Browning et al. (1993) 72: 847; Browning et al. (1995) 154:33; Matsumoto et al.(1997) Immunol. Rev. 156:137). Activation of LT-β-R has been shown to induce the apoptotic death of certain cancer cell lines in vivo (PCT/US96/01386). Methods of enhancing the anti-tumor effects of LT-β-R activating agents, such as specific humanized anti-LT-β-R antibodies, would be useful for treating or reducing the advancement, severity or effects of neoplasia in subjects (e.g., humans).

### SUMMARY OF THE INVENTION

The present invention provides, in part, methods and articles of manufacture for the treatment of cancer. More specifically, it has been shown that the use of a lymphotoxin-beta receptor (LT-β-R) binding molecule, e.g., an anti-LT-β-R antibody, and at least one additional agent, which is not a lymphotoxin receptor binding molecule, (e.g., an agent that inhibits angiogenesis, or a biologic agent) is more effective at reducing the size of certain tumors, e.g., solid tumors, than either agent alone. As shown herein, treatment of established solid tumors with a combination therapy of the invention produces a meaningful tumor growth inhibition (% inhibition) compared to treatment of the tumor with either agent alone. Furthermore, it has been demonstrated that the combination of antibody and second agent is more effective at decreasing vascularization of a solid tumor and/or increasing the permeability of a solid tumor.

Moreover, the combination therapies of the invention have additional benefits. In one embodiment of the invention, the combination therapy of the invention has an improved safety profile. In another embodiment, a combination therapy of the invention allows for either or both of the components of the combination therapy to be used at a dose lower than that at which they are used alone.

Accordingly, in one aspect the present invention provides a method for reducing tumor size in a subject having a tumor of a size greater than about 2 mm×2 mm, comprising administering an anti-lymphotoxin-beta receptor (LT-β-R) binding molecule and at least one additional agent to the subject, such that the tumor size is reduced.

The invention also provides a method for decreasing vascularization of a solid tumor in a subject having a solid tumor, comprising administering an anti-lymphotoxin-beta receptor (LT-β-R) binding molecule and at least one additional agent to the subject, such that vascularization of the solid tumor is decreased.

The invention also provides a method for increasing permeability of a solid tumor in a subject having a solid tumor, comprising administering an anti-lymphotoxin-beta receptor (LT-β-R) binding molecule and at least one additional agent to the subject, such that permeability of the solid tumor to the anti-LT-β-R antibody is increased.

The invention also includes a method of treating cancer, comprising sensitizing tumor cells with an anti-LT-β-R binding molecule and administering a chemotherapeutic agent and at least one additional agent.

The at least one additional agent can be administered to the subject prior to administration of the anti-LT-β-R binding molecule or the at least one additional agent can be administered to the subject concomitantly with the administration of the anti-LT-β-R binding molecule.

In one embodiment, the at least one additional agent inhibits angiogenesis. In one embodiment, the at least one additional agent is a biologic agent. In one embodiment, the biologic agent that inhibits angiogenesis is an antibody or antigen binding fragment thereof. In another embodiment, the biologic agent is an anti-VEGF antibody. In one embodiment, the anti-VEGF antibody is bevacizumab. In another embodiment, the biologic agent is an anti-EGFR antibody. In one embodiment, the anti-EGFR antibody is cetuximab.

In yet another embodiment, the biologic agent is selected from the group consisting of rituximab, trastuzumab, tositumomab, ibritumomab, alelmtuzumab, epratuzumab, gemtuzumab ozogamicin, oblimersen, and panitumumab.

In one embodiment, the biologic agent is an interferon or an interleukin.

In one embodiment of the invention, the LT-β-R binding molecule is a humanized binding molecule. In one embodiment of the invention, the humanized binding molecule is humanized CBE11.

In another embodiment of the invention, the anti-LT-β-R binding molecule is a multivalent anti-LT-β-R binding molecule. In one embodiment, the multivalent anti-LT-β-R binding molecule comprises at least one antigen binding site derived from the CBE11 antibody.

In yet another embodiment, the anti-LT-β-R binding molecule is conjugated to a chemotherapeutic agent or an immunotoxin.

In one embodiment of the invention, the tumor is a carcinoma, e.g., an adenocarcinoma or a squamous cell carcinoma.

In another embodiment, the tumor is selected from the group consisting of a colon tumor, a cervical tumor, a gastric tumor, or a pancreatic tumor.

In yet another embodiment, the tumor is at a stage selected from the group consisting of Stage I, Stage II, Stage III, and Stage IV.

In one embodiment, the tumor is at least about 1 mm×1 mm. In another embodiment, the tumor is at least about 2 mm×2 mm. In yet another embodiment, the tumor has a volume of at least about 1 cm³.

In one embodiment, the tumor is metastatic.

In one embodiment, the methods of the invention further comprise administering a chemotherapeutic agent to the subject. In one embodiment, the chemotherapeutic agent is selected from the group consisting of gemcitabine, adriamycin, Camptosar, carboplatin, cisplatin, and Taxol.

The present invention provides a method for reducing tumor size in a subject having a tumor of a size greater than about 2 mm×2 mm, comprising administering an anti-lymphotoxin-beta receptor (LT-β-R) binding molecule and at least one additional agent that inhibits angiogenesis to the subject, such that the tumor size is reduced.

The invention also provides a method for decreasing vascularization of a solid tumor in a subject having a solid tumor, comprising administering an anti-lymphotoxin-beta receptor (LT-β-R) binding molecule and at least one additional agent that inhibits angiogenesis to the subject, such that vascularization of the solid tumor is decreased.

The invention also provides a method for increasing permeability of a solid tumor in a subject having a solid tumor, comprising administering an anti-lymphotoxin-beta receptor (LT-β-R) binding molecule and at least one additional agent that inhibits angiogenesis to the subject, such that permeability of the solid tumor to the anti-LT-β-R binding molecule is increased.

The at least one additional agent that inhibits angiogenesis can be administered to the subject prior to administration of the anti-LT-β-R binding molecule or the at least one additional agent that inhibits angiogenesis can be administered to the subject concomitantly with the anti-LT-β-R binding molecule.

In one embodiment, the at least one additional agent that inhibits angiogenesis is a biologic agent. In one embodiment, the biologic agent that inhibits angiogenesis is selected from the group consisting of gefitinib, imatinib mesylate, and bortezomib.

In one embodiment of the invention, the LT-β-R binding molecule is a humanized binding molecule. In one embodiment of the invention, the humanized binding molecule is humanized CBE11. In another embodiment of the invention, the anti-LT-β-R binding molecule is a multivalent anti-LT-β-R binding molecule. In one embodiment, the multivalent anti-LT-β-R binding molecule comprises at least one antigen binding site derived from the CBE11 antibody.

In yet another embodiment, the anti-LT-β-R binding molecule is conjugated to a chemotherapeutic agent or an immunotoxin.

In one embodiment of the invention, the tumor is a carcinoma, e.g., an adenocarcinoma or a squamous cell carcinoma.

In another embodiment, the tumor is selected from the group consisting of a colon tumor, a cervical tumor, a gastric tumor, or a pancreatic tumor.

In yet another embodiment, the tumor is at a stage selected from the group consisting of Stage I, Stage II, Stage III, and Stage IV.

In one embodiment, the tumor is at least about 1 mm×1 mm. In another embodiment, the tumor is at least about 2 mm×2 mm. In yet another embodiment, the tumor has a volume of at least about 1 cm³.

In one embodiment, the tumor is metastatic.

In one embodiment, the methods of the invention further comprise administering a chemotherapeutic agent to the subject. In one embodiment, the chemotherapeutic agent is selected from the group consisting of gemcitabine, adriamycin, Camptosar, carboplatin, cisplatin, and Taxol.

In one embodiment, the administration of an anti-lymphotoxin-beta receptor (LT-β-R) binding molecule, or an antigen-binding fragment thereof, and at least one agent that inhibits angiogenesis results in a % tumor inhibition of about 58% or greater.

The present invention provides a method for reducing tumor size in a subject having a colon tumor of a size greater than about 2 mm×2 mm, comprising administering a humanized CBE11 antibody (huCBE11) and bevacizumab to the subject, such that the tumor size is reduced.

The present invention also provides a method for reducing tumor size in a subject having a colon tumor of a size greater than about 2 mm×2 mm, comprising administering an anti-lymphotoxin-beta receptor (LT-β-R) binding molecule and at least one EGFR inhibiting agent to the subject, such that the tumor size is reduced.

In one embodiment, the EGFR inhibiting agent is cetuximab or erlotinib.

In one embodiment, the anti-LT-β-R binding molecule is huCBE11.

The invention further provides an article of manufacture comprising, a packaging material, an anti-lymphotoxin-beta receptor (LT-β-R) binding molecule, and a label or package insert contained within the packaging material indicating that the anti-LT-β-R binding molecule can be administered with at least one additional agent.

The present invention also provides an article of manufacture comprising, a packaging material, a second agent, and a label or package insert contained within the packaging material indicating that the at least one additional agent can be administered with an anti-lymphotoxin-beta receptor (LT-β-R) binding molecule.

In one embodiment, the at least one additional agent in the article of manufacture is an agent that inhibits angiogenesis. In one embodiment, the agent in the article of manufacture is a biologic agent. In one embodiment, the biologic agent in the article of manufacture is bevacizumab or cetuximab.

In one embodiment, the anti-LT-β-R binding molecule in the article of manufacture is huCBE11.

The present invention also provides an article of manufacture comprising, a packaging material, a huCBE11 antibody, and a label or package insert contained within the packaging material indicating that the huCBE11 antibody can be administered with bevacizumab or cetuximab.

The present invention also provides an article of manufacture comprising, a packaging material, bevacizumab or cetuximab, and a label or package insert contained within the packaging material indicating that the biologic agent can be administered with a huCBE11 antibody.

Other features and advantages of the invention will be apparent from the following detailed description, and from the claims.

### BRIEF DESCRIPTION OF THE FIGURES

FIG. 1 depicts a graph showing the effect of huCBE11 at 0.2 mg/kg, 2 mg/kg, 4 mg/kg, and 20 mg/kg against tumor weight (length×width²/2) in the KM-20L2 human colon adenocarcinoma model over the course of treatment, as compared to a vehicle control. Treatment was initiated when the xenograft tumor was approximately 65 mg. The first dose is indicated by an arrow.

FIG. 2 depicts a graph showing the effect of bevacizumab (Avastin) 1 mg/kg, 2 mg/kg, and 4 mg/kg against tumor weight (length×width²/2) in the KM-20L2 human colon adenocarcinoma model over the course of treatment, as compared to a vehicle control. Treatment was initiated when the xenograft tumor was approximately 75 mg. The first dose is indicated by an arrow.

FIG. 3 depicts a graph showing the effect of bevacizumab (Avastin) 1 mg/kg, 2 mg/kg, and 4 mg/kg against tumor weight (length×width²/2) in the KM-20L2 human colon adenocarcinoma model over the course of treatment, as compared to a vehicle control. Treatment was initiated when the xenograft tumor was approximately 100 mg. The first dose is indicated by an arrow.

FIG. 4 depicts a graph showing the effect of bevacizumab (Avastin) in combination with huCBE11 against tumor weight (length×width²/2) in the KM-20L2 human colon adenocarcinoma model over the course of treatment, as compared to a vehicle control. Treatment was initiated when the xenograft tumor was approximately 65 mg. The first dose of each agent is indicated by an arrow.

FIG. 5 depicts a scatter plot showing the effect of bevacizumab (Avastin) in combination with huCBE11 against tumor weight (length×width 2/2) in the KM-20L2 human colon adenocarcinoma model at day 51 of the study, as compared to a vehicle control. Treatment was initiated when the xenograft tumor was approximately 65 mg.

FIG. 6 depicts a graph showing the assessment of tumor growth inhibition (% T/C) of bevacizumab (Avastin) in combination with huCBE11 in the KM-20L2 human colon adenocarcinoma model. Treatment was initiated when the xenograft tumor was approximately 65 mg.

FIG. 7 depicts a graph showing the effect of bevacizumab (Avastin) in combination with huCBE11 against tumor weight (length×width²/2) in the KM-20L2 human colon adenocarcinoma model over the course of treatment, as compared to a vehicle control. Treatment was initiated when the xenograft tumor was approximately 200 mg. The first dose of each agent is indicated by an arrow.

FIG. 8 depicts a scatter plot showing the effect of bevacizumab (Avastin) in combination with huCBE11 against tumor weight (length×width²/2) in the KM-20L2 human colon adenocarcinoma model at day 57 of the study, as compared to a vehicle control. Treatment was initiated when the xenograft tumor was approximately 200 mg.

FIG. 9 depicts a graph showing the assessment of tumor growth inhibition (% T/C) of bevacizumab (Avastin) in combination with huCBE11 in the KM-20L2 human colon adenocarcinoma model. Treatment was initiated when the xenograft tumor was approximately 200 mg.

FIG. 10 depicts a graph showing the effect of huCBE11 at 0.2 mg/kg, 2 mg/kg, 4 mg/kg, and 20 mg/kg against tumor weight (length×width²/2) in the WiDr adrenocarcinoma model over the course of treatment, as compared to a vehicle control. Treatment was initiated when the xenograft tumor was approximately 65 mg. The first dose is indicated by an arrow.

FIG. 11 depicts a graph showing the effect of bevacizumab (Avastin) 0.25 mg/kg, 0.5 mg/kg, 1 mg/kg, 2 mg/kg, 4 mg/kg, and 8 mg/kg against tumor weight (length×width²/2) in the WiDr adrenocarcinoma model over the course of treatment, as compared to a vehicle control. Treatment was initiated when the xenograft tumor was approximately 100 mg. The first dose is indicated by an arrow.

FIG. 12 depicts a graph showing the effect of bevacizumab (Avastin) in combination with huCBE11 against tumor weight (length×width²/2) in the WiDr adrenocarcinoma model over the course of treatment, as compared to a vehicle control. Treatment was initiated when the xenograft tumor was approximately 65 mg. The first dose of each agent is indicated by an arrow.

FIG. 13 depicts a scatter plot showing the effect of bevacizumab (Avastin) in combination with huCBE11 against tumor weight (length×width²/2) in the WiDr adrenocarcinoma model at day 54 of the study, as compared to a vehicle control. Treatment was initiated when the xenograft tumor was approximately 65 mg.

FIG. 14 depicts a graph showing the assessment of tumor growth inhibition (% T/C) of bevacizumab (Avastin) in combination with huCBE11 in the WiDr adrenocarcinoma model. Treatment was initiated when the xenograft tumor was approximately 65 mg.

FIG. 15 depicts a graph showing the effect of bevacizumab (Avastin) in combination with huCBE11 against tumor weight (length×width²/2) in the WiDr adrenocarcinoma model over the course of treatment, as compared to a vehicle control. Treatment was initiated when the xenograft tumor was approximately 200 mg. The first dose of each agent is indicated by an arrow.

FIG. 16 depicts a scatter plot showing the effect of bevacizumab (Avastin) in combination with huCBE11 against tumor weight (length×width²/2) in the WiDr adrenocarcinoma model at day 54 of the study, as compared to a vehicle control. Treatment was initiated when the xenograft tumor was approximately 200 mg.

FIG. 17 depicts a graph showing the assessment of tumor growth inhibition (% T/C) of bevacizumab (Avastin) in combination with huCBE11 in the WiDr adrenocarcinoma model. Treatment was initiated when the xenograft tumor was approximately 200 mg.

### DETAILED DESCRIPTION OF THE INVENTION

### 1. Definitions

For convenience, before further description of the present invention, certain terms employed in the specification, examples and appended claims are defined here.

The singular forms “a”, “an”, and “the” include plural references unless the context clearly dictates otherwise.

The term “administering” includes any method of delivery of a pharmaceutical composition or therapeutic agent into a subject's system or to a particular region in or on a subject. The phrases “systemic administration,” “administered systemically”, “peripheral administration”, and “administered peripherally” as used herein mean the administration of a compound, drug or other material other than directly into the central nervous system, such that it enters the subject's system and, thus, is subject to metabolism and other like processes, for example, subcutaneous administration. “Parenteral administration” and “administered parenterally” means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intra-articular, subcapsular, subarachnoid, intraspinal and intrasternal injection and infusion.

The term “lymphotoxin 13 receptor” (“LT-β-R”) refers to the art known member of the tumor necrosis factor (TNF) superfamily of molecules which mediates a wide range of innate and adaptive immune response functions (for a review, see, e.g., Gommerman and Browning (2003) Nat Rev 3:642, the contents of which are incorporated by reference).

The term “binding molecule” refers to a molecule that comprises at least one binding domain which comprises a binding site that specifically binds to a target molecule (such as an antigen). For example, in one embodiment, a binding molecule for use in the methods of the invention comprises an immunoglobulin antigen binding site or the portion of a ligand molecule that is responsible for receptor binding.

In one embodiment, the binding molecule comprises at least two binding sites. In one embodiment, the binding molecules comprise two binding sites. In one embodiment, the binding molecules comprise three binding sites. In another embodiment, the binding molecules comprise four binding sites.

The term “LT-β-R binding molecule” refers to a molecule that comprises at least one lymphotoxin beta receptor (LT-β-R) binding site. Examples of LT-β-R binding molecules which can be used in the methods and articles of manufacture of the invention include, but are not limited to, binding molecules described in WO 96/22788, WO 02/30986, and WO 04/002431, each of which is incorporated in its entirety by reference herein.

In one embodiment, the binding molecules of the invention are “antibody” or “immunoglobulin” molecules, e.g., naturally occurring antibody or immunoglobulin molecules or genetically engineered antibody molecules that bind antigen in a manner similar to antibody molecules. As used herein, the term “immunoglobulin” includes a polypeptide having a combination of two heavy and two light chains whether or not it possesses any relevant specific immunoreactivity. “Antibodies” refers to such assemblies which have significant known specific immunoreactive activity to an antigen. Antibodies and immunoglobulins comprise light and heavy chains, with or without an interchain covalent linkage between them. Basic immunoglobulin structures in vertebrate systems are relatively well understood.

The generic term “immunoglobulin” comprises five distinct classes of antibody that can be distinguished biochemically. All five classes of antibodies are clearly within the scope of the present invention, the following discussion will generally be directed to the IgG class of immunoglobulin molecules. With regard to IgG, immunoglobulins comprise two identical light polypeptide chains of molecular weight approximately 23,000 Daltons, and two identical heavy chains of molecular weight 53,000-70,000. The four chains are joined by disulfide bonds in a “Y” configuration wherein the light chains bracket the heavy chains starting at the mouth of the “Y” and continuing through the variable region.

Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. By convention the numbering of the constant region domains increases as they become more distal from the antigen binding site or amino-terminus of the antibody. The N-terminus is a variable region and at the C-terminus is a constant region; the CH3 and CL domains actually comprise the carboxy-terminus of the heavy and light chain, respectively.

Light chains are classified as either kappa or lambda (κ, λ). Each heavy chain class may be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain. Those skilled in the art will appreciate that heavy chains are classified as gamma, mu, alpha, delta, or epsilon, (γ, μ, α, δ, ε) with some subclasses among them (e.g., γ1-γ4). It is the nature of this chain that determines the “class” of the antibody as IgG, IgM, IgA IgG, or IgE, respectively. The immunoglobulin subclasses (isotypes) e.g., IgG₁, IgG₂, IgG₃, IgG₄, IgA₁, etc. are well characterized and are known to confer functional specialization. Modified versions of each of these classes and isotypes are readily discernable to the skilled artisan in view of the instant disclosure and, accordingly, are within the scope of the instant invention.

The variable region allows the antibody to selectively recognize and specifically bind epitopes on antigens. That is, the V_{L} domain and V_{H} domain of an antibody combine to form the variable region that defines a three dimensional antigen binding site. This quaternary antibody structure forms the antigen binding site present at the end of each arm of the Y. More specifically, the antigen binding site is defined by three complementary determining regions (CDRs) on each of the V_{H} and V_{L} chains.

The term “antibody”, as used herein, includes whole antibodies, e.g., of any isotype (IgG, IgA, IgM, IgE, etc.), and includes antigen binding fragments thereof. Exemplary antibodies include monoclonal antibodies, polyclonal antibodies, chimeric antibodies, humanized antibodies, human antibodies, and multivalent antibodies. Antibodies may be fragmented using conventional techniques. Thus, the term antibody includes segments of proteolytically-cleaved or recombinantly-prepared portions of an antibody molecule that are capable of actively binding to a certain antigen. Non-limiting examples of proteolytic and/or recombinant antigen binding fragments include Fab, F(ab′)2, Fab′, Fv, and single chain antibodies (sFv) containing a V[L] and/or V[H] domain joined by a peptide linker.

As used herein, the term “humanized antibody” refers to an antibody or antibody construct in which the complementarity determining regions (CDRs) of an antibody from one species have been grafted onto the framework regions of the variable region of a human. Such antibodies may or may not include framework mutations, backmutations, and/or CDR mutations to optimize antigen binding.

The term “multispecific” includes binding molecules having specificity for more than one target antigen. Such molecules have more than one binding site where each binding site specifically binds (e.g., immunoreacts with) a different target molecule or a different antigenic site on the same target.

In one embodiment, a multispecific binding molecule of the invention is a bispecific molecule (e.g., antibody, minibody, domain deleted antibody, or fusion protein) having binding specificity for at least two targets, e.g., more than one target molecule or more than one epitope on the same target molecule.

In one embodiment, modified forms of antibodies can be made from a whole precursor or parent antibody using techniques known in the art. Exemplary techniques are discussed in more detail below. In particularly preferred embodiments both the variable and constant regions of polypeptides of the invention are human. In one embodiment, fully human antibodies can be made using techniques that are known in the art. For example, fully human antibodies against a specific antigen can be prepared by administering the antigen to a transgenic animal which has been modified to produce such antibodies in response to antigenic challenge, but whose endogenous loci have been disabled. Exemplary techniques that can be used to make antibodies are described in U.S. Pat. Nos. 6,150,584; 6,458,592; 6,420,140. Other techniques are known in the art.

In one embodiment, a binding molecule of the invention comprises an antibody molecule, e.g., an intact antibody molecule, or a fragment of an antibody molecule. In another embodiment, binding molecule of the invention is a modified or synthetic antibody molecule. In one embodiment, a binding molecule of the invention comprises all or a portion of (e.g., at least one antigen binding site from, at least one CDR from) a monoclonal antibody, a humanized antibody, a chimeric antibody, or a recombinantly produced antibody.

In embodiments where the binding molecule is an antibody or modified antibody, the antigen binding site and the heavy chain portions need not be derived from the same immunoglobulin molecule. In this regard, the variable region may be derived from any type of animal that can be induced to mount a humoral response and generate immunoglobulins against the desired antigen. As such, the variable region of the polypeptides may be, for example, of mammalian origin e.g., may be human, murine, non-human primate (such as cynomolgus monkeys, macaques, etc.), lupine, camelid (e.g., from camels, llamas and related species). In another embodiment, the variable region may be condricthoid in origin (e.g., from sharks).

In one embodiment, the binding molecules of the invention are modified antibodies. As used herein, the term “modified antibody” includes synthetic forms of antibodies which are altered such that they are not naturally occurring, e.g., antibodies that do not comprise complete heavy chains (such as, domain deleted antibodies or minibodies); multispecific forms of antibodies (e.g., bispecific, trispecific, etc.) altered to bind to two or more different antigens or to different epitopes on a single antigen); heavy chain molecules joined to scFv molecules and the like. ScFv molecules are known in the art and are described, e.g., in U.S. Pat. No. 5,892,019. In addition, the term “modified antibody” includes multivalent forms of antibodies (e.g., trivalent, tetravalent, etc., antibodies that bind to three or more copies of the same antigen).

In one embodiment, the term, “modified antibody” according to the present invention includes immunoglobulins, antibodies, or immunoreactive fragments or recombinants thereof, in which at least a fraction of one or more of the constant region domains has been deleted or otherwise altered so as to provide desired biochemical characteristics such as the ability to non-covalently dimerize, increased ability to localize at the site of a tumor, or reduced serum half-life when compared with a whole, unaltered antibody of approximately the same immunogenicity. In a preferred embodiment, the polypeptides of the present invention are domain deleted antibodies which comprise a polypeptide chain similar to an immunoglobulin heavy chain, but which lack at least a portion of one or more heavy chain domains. More preferably, one entire domain of the constant region of the modified antibody will be deleted and even more preferably all or part of the CH2 domain will be deleted.

In preferred embodiments, a binding molecule of the invention will not elicit a deleterious immune response in a human. Modifications to the constant region compatible with the instant invention comprise additions, deletions or substitutions of one or more amino acids in one or more domains. That is, the binding molecules of the invention may comprise alterations or modifications to one or more of the three heavy chain constant domains (CH1, CH2 or CH3) and/or to the light chain constant region domain (CL).

In one embodiment, the binding molecules of the invention may be modified to reduce their immunogenicity using art-recognized techniques. For example, antibodies or polypeptides of the invention can be humanized, deimmunized, or chimeric antibodies can be made. These types of antibodies are derived from a non-human antibody, typically a murine antibody, that retains or substantially retains the antigen-binding properties of the parent antibody, but which is less immunogenic in humans. This may be achieved by various methods, including (a) grafting the entire non-human variable domains onto human constant regions to generate chimeric antibodies; (b) grafting at least a part of one or more of the non-human complementarity determining regions (CDRs) into a human framework and constant regions with or without retention of critical framework residues; or (c) transplanting the entire non-human variable domains, but “cloaking” them with a human-like section by replacement of surface residues. Such methods are disclosed in Morrison et al., Proc. Natl. Acad. Sci. 81: 6851-5 (1984); Morrison et al., Adv. Immunol. 44: 65-92 (1988); Verhoeyen et al., Science 239: 1534-1536 (1988); Padlan, Molec. Immun. 28: 489-498 (1991); Padlan, Molec. Immun. 31: 169-217 (1994), and U.S. Pat. Nos. 5,585,089, 5,693,761 and 5,693,762 all of which are hereby incorporated by reference in their entirety.

An “agent that inhibits angiogenesis” is any agent that inhibits, for example, the initiation of blood vessel formation, the development of a blood vessel, and/or the maintenance of a blood vessel.

In one embodiment an agent that inhibits angiogenesis is a biologic agent.

The term “biologic” or “biologic agent” refers to any pharmaceutically active agent made from living organisms and/or their products which is intended for use as a therapeutic. In one embodiment of the invention, biologic agents which can be used in combination with an anti-LT-β-R binding molecule include, but are not limited to e.g., antibodies, nucleic acid molecules, e.g., antisense nucleic acid molecules, polypeptides or proteins. Such biologics can be administered in combination with an anti-LT-β-R binding molecule by administration of the biologic agent, e.g., prior to the administration of the anti-LT-βR binding molecule, concomitantly with the anti-LT-βR binding molecule, or after the anti-LT-βR binding molecule.

In one embodiment, cells from a subject can be contacted in vitro with the anti-LT-βR binding molecule and/or the biologic agent and then introduced into the subject. The subject may then be treated with the second phase of the combination therapy, e.g., the anti-LT-βR binding molecule and/or the biologic agent.

The term “combination therapy”, as used herein, refers to a therapeutic regimen comprising, e.g., an anti-LTβR binding molecule and a second agent, e.g., an agent that inhibits angiogenesis or a biologic agent. The anti-LTβR binding molecule and the second agent may be formulated for separate administration or may be formulated for administration together.

The term “cancer” or “neoplasia” refers in general to any malignant neoplasm or spontaneous growth or proliferation of cells. A subject having “cancer”, for example, may have a leukemia, lymphoma, or other malignancy of blood cells. In certain embodiments, the subject methods are used to treat a solid tumor. Exemplary solid tumors include but are not limited to non small cell lung cancer (NSCLC), testicular cancer, lung cancer, ovarian cancer, uterine cancer, cervical cancer, pancreatic cancer, colorectal cancer (CRC), breast cancer, as well as prostate, gastric, skin, stomach, esophageal, and bladder cancer. In one embodiment of the invention, a solid tumor is a colon tumor. In another embodiment of the invention, a solid tumor is selected from the group consisting of a colon tumor, a cervical tumor, a gastric tumor, and a pancreatic tumor.

In certain embodiments of the invention, the subject methods are used to treat (e.g., reduce tumor size, decrease the vascularization, and/or increase the permeability of) an established tumor. As used herein, an “established tumor” is a solid tumor of sufficient size such that nutrients, i.e., oxygen can no longer permeate to the center of the tumor from the subject's vasculature by osmosis and therefore the tumor requires its own vascular supply to receive nutrients.

In one embodiment, the subject methods are used to treat a vascularized tumor. A vascularized tumor includes tumors having the hallmarks of established vasculature. Such tumors are identified by their size and/or by the presence of markers of vessels or angiogenesis.

In another embodiment, the subject methods are used to treat a solid tumor that is not quiescent and is actively undergoing exponential growth.

The term “carcinoma” refers to any of various types of malignant neoplasias derived from epithelial cells, e.g., glandular cells (“adenoma” or “adenocarcinoma”) or squamous cells (“squamous cell carcinoma”). Carcinomas often infiltrate into adjacent tissue and spread (“metastasize”) to distant organs, e.g., bone, liver, lung or brain. As used herein, “cervical cancer” refers to a tumor that arises in the cervix, i.e., the lower, narrow part of the uterus or womb. As used herein, the term cervical cancer includes squamous cell carcinomas, adenocarcinomas, and mixed carcinomas, i.e., adenosquamous carcinomas, of the cervix.

Based on the FIGO system, cervical cancer can be “Stage 0-IV”. “Stage 0”, also referred to as “carcinoma in situ”, is a tumor found only in the epithelial cells lining the cervix and which has not invaded deeper tissues. “Stage I” cervical cancer is a tumor strictly confined to the cervix. In “Stage IA”, a very small amount of tumor can be seen under a microscope. In “Stage IA1”, the tumor has penetrated an area less than 3 millimeters deep and less than 7 millimeters wide. In “Stage IA2”. The tumor has penetrated an area 3 to 5 millimeters deep and less than 7 millimeters wide. In “Stage IB” the tumor can be seen without a microscope. Stage IB also includes tumors that cannot be seen without a microscope but that are more than 7 millimeters wide and have penetrated more than 5 millimeters of connective cervical tissue. “Stage IB1” is a tumor that is no bigger than 4 centimeters. “Stage IB2” tumors are bigger than 4 centimeters and have has spread to organs and tissues outside the cervix but are still limited to the pelvic area. “Stage II” cervical cancer refers to a tumor extending beyond the cervix and/or the upper two-thirds of the vagina, but not onto the pelvic wall. In “Stage IIA”, the tumor has spread beyond the cervix to the upper part of the vagina. In “Stage IIB”, the tumor has spread to the tissue next to the cervix. “Stage III” cervical cancer refers to a tumor that has spread to the lower third of the vagina or onto the pelvic wall; the tumor may block the flow of urine from the kidneys to the bladder. In “Stage IIIA”, the tumor has spread to the lower third of the vagina. In “Stage IIIB”, the tumor has spread to the pelvic wall and/or blocks the flow of urine from the kidneys to the bladder. “Stage IV” cervical cancer refers to a tumor that has spread (metastasized) to other parts of the body, i.e., the bladder or rectum (“Stage IVA”), or elsewhere, e.g., the liver or lungs (“Stage IVB”).

As used herein, “colon cancer” or “colorectal cancer” refers to a tumor that arises from the inner lining of the large intestine, or colon. Most, if not all, of these cancers develop from colonic polyps. The term “colon cancer” also refers to carcinomas, lymphomas, carcinoid tumors, melanomas, and sarcomas of the colon.

Colorectal cancer can be divided into Stages 0-IV. “Stage 0” colorectal cancer is found only in the innermost lining of the colon or rectum. Carcinoma in situ is another name for Stage 0 colorectal cancer. “Stage I” colorectal cancer refers to a tumor that has grown into the inner wall of the colon or rectum. The tumor has not reached the outer wall of the colon or extended outside the colon. “Dukes' A” is another name for Stage I colorectal cancer. In “Stage II” colorectal cancer, the tumor extends more deeply into or through the wall of the colon or rectum. It may have invaded nearby tissue, but cancer cells have not spread to the lymph nodes. “Dukes' B” is another name for Stage II colorectal cancer. “Stage III” colorectal cancer refers to a tumor that has spread to nearby lymph nodes, but not to other parts of the body. “Dukes' C” is another name for Stage III colorectal cancer. In “Stage IV” colorectal cancer, the tumor has spread to other parts of the body, such as the liver or lungs. “Dukes' D” is another name for Stage IV colorectal cancer.

As used herein “gastrointestinal cancer” or “GI cancer” is a cancer of any of the gastrointestinal tract organs or organs of the alimentary canal, i.e., mouth, esophagus, stomach, duodenum, small intestine, large intestine or colon, rectum, and anus.

The term “gastric cancer” or “gastric neoplasia”, also referred to as “stomach cancer”, as used herein, includes adenocarcinomas, lymphomas, stromal tumors, squamous cell tumors, adenosquamous carcinomas, carcinoids, and leiomyosarcomas of the stomach. Gastric cancer, as used herein, also refers to tumors that occur in the lining of the stomach (mucosa), tumors that develop in the lower part of the stomach (pylorus), the middle part (body) of the stomach, those that develop in the upper part (cardia) of the stomach, as well as those tumors that develop in more than one part of the stomach. Gastric cancer may be “metastatic” from another source (e.g., colon) or may be “primary” (a tumor of stomach cell origin). For example, gastric cancer can metastasize to the esophagus or the small intestine, and can extend through the stomach wall to nearby lymph nodes and organs (e.g., liver, pancreas, and colon). Gastric cancer can also metastasize to other parts of the body (e.g., lungs, ovaries, bones).

Gastric cancer can be Stage 0-IV. “Stage 0” gastric cancer, also referred to as “carcinoma in situ”, is a tumor found only in the inside lining of the mucosal layer of the stomach wall. “Stage I gastric cancer” is divided into “Stage IA” and “Stage IB”, depending on where the cancer has spread. In Stage IA, the cancer has spread completely through the mucosal layer of the stomach wall. In Stage IB, the cancer has spread completely through the mucosal layer of the stomach wall and is found in up to 6 lymph nodes near the tumor; or to the muscularis layer of the stomach wall. In “Stage II gastric cancer”, cancer has spread completely through the mucosal layer of the stomach wall and is found in 7 to 15 lymph nodes near the tumor; or to the muscularis layer of the stomach wall and is found in up to 6 lymph nodes near the tumor; or to the serosal layer of the stomach wall but not to lymph nodes or other organs. “Stage III gastric cancer” is divided into “Stage IIIA” and “Stage IIIB” depending on where the cancer has spread. Stage IIIA refers to cancer that has spread to the muscularis layer of the stomach wall and is found in 7 to 15 lymph nodes near the tumor; or the serosal layer of the stomach wall and is found in 1 to 6 lymph nodes near the tumor; or organs next to the stomach but not to lymph nodes or other parts of the body. Stage IIIB refers to cancer that has spread to the serosal layer of the stomach wall and is found in 7 to 15 lymph nodes near the tumor. In “Stage IV gastric cancer”, cancer has spread to organs next to the stomach and to at least one lymph node; or more than 15 lymph nodes; or other parts of the body.

As used herein, the term “pancreatic cancer” refers to tumor arising in the pancreas, and includes “ductal adenocarcinomas” and “islet cell carcinomas”.

Pancreatic cancer can be “Stage I-IV”. In “Stage I” pancreatic cancer, the cancer is confined to the pancreas and is often referred to as being “resectable”. In “Stage IA”, the tumor is confined to the pancreas and is less than 2 cm in size; it has not spread to nearby lymph nodes or distant sites. In “Stage IB” the tumor is confined to the pancreas and is larger than 2 cm in size and has not spread to nearby lymph nodes or distant sites. “Stage II” pancreatic cancer is no longer resectable. In “Stage IIA”, the tumor has grown outside of the pancreas but not into organs immediately adjacent to the pancreas, such as the bile duct or the duodenum, and has not spread to nearby lymph nodes. In “Stage IIB”, the tumor is either confined to the pancreas or growing outside the pancreas but not into organs immediately adjacent to pancreas, such as the bile duct or the duodenum, but it has spread to nearby lymph nodes. In “Stage III”, the tumor has grown outside the pancreas into nearby organs such as the colon, stomach, or spleen, and may or may not have spread to nearby lymph nodes. In “Stage IV” the tumor has spread to other parts of the body, such as the liver or lungs.

The term “chemotherapeutic agent” refers to a molecule or composition used to treat malignancy. Such agents may be used in combination with an anti-LT-βR binding molecule or with a combination therapy of the invention. Chemotherapeutic agents include agents that can be conjugated to an anti-LT-βR binding molecule and/or may be used in combination with the combination therapy in unconjugated form. Exemplary chemotherapeutic agents are discussed below.

The term “effective amount” refers to that amount of combination therapy which is sufficient to affect a desired result on a cancerous cell or tumor, including, but not limited to, for example, reducing tumor size, reducing tumor volume, decreasing vascularization of a solid tumor and/or increasing the permeability of a solid tumor to an agent, either in vitro or in vivo. In certain embodiments of the invention, an effective amount of a combination therapy is the amount that results in a % tumor inhibition of more than about 58%, 60%, 65%, 70%, 75%, 80%, 85%, 90%, 95%, 100%. The term also includes that amount of a combination therapy which is sufficient to achieve a desired clinical result, including but not limited to, for example, ameliorating disease, stabilizing a patient, preventing or delaying the development of, or progression of cancer in a patient. An effective amount of the combination therapy can be determined based on one administration or repeated administration. Methods of detection and measurement of the indicators above are known to those of skill in the art. Such methods include, but are not limited to measuring reduction in tumor burden, reduction of tumor size, reduction of tumor volume, reduction in proliferation of secondary tumors, decreased solid tumor vascularization, expression of genes in tumor tissue, presence of biomarkers, lymph node involvement, histologic grade, and nuclear grade.

In one embodiment of the invention, tumor burden is determined. “Tumor burden” also referred to as “tumor load”, refers to the total amount of tumor material distributed throughout the body. Tumor burden refers to the total number of cancer cells or the total size of tumor(s), throughout the body, including lymph nodes and bone barrow. Tumor burden can be determined by a variety of methods known in the art, such as, e.g. by measuring the dimensions of tumor(s) upon removal from the subject, e.g., using calipers, or while in the body using imaging techniques, e.g., ultrasound, computed tomography (CT) or magnetic resonance imaging (MRI) scans.

In one embodiment of the invention, tumor size is determined. The term “tumor size” refers to the total size of the tumor which can be measured as the length and width of a tumor. Tumor size may be determined by a variety of methods known in the art, such as, e.g. by measuring the dimensions of tumor(s) upon removal from the subject, e.g., using calipers, or while in the body using imaging techniques, e.g., ultrasound, computed tomography (CT) or magnetic resonance imaging (MRI) scans.

In one embodiment of the invention, tumor size is determined by determining tumor weight. In one embodiment, tumor weight is determined by measuring the length of the tumor, multiplying it by the square of the width of the tumor, and dividing that sum by 2 (as described in the Examples section below).

In one embodiment of the invention, tumor size is determined by determining tumor volume. The term “tumor volume” refers to the total size of the tumor, which includes the tumor itself plus affected lymph nodes if applicable. Tumor volume may be determined by a variety of methods known in the art, such as, e.g. by measuring the dimensions of tumor(s) upon removal from the subject, e.g., using calipers, or while in the body using an imaging techniques, e.g., ultrasound, computed tomography (CT) or magnetic resonance imaging (MRI) scans, and calculating the volume using equations based on, for example, the z-axis diameter, or on standard shapes such as the sphere, ellipsoid, or cube. In one embodiment, tumor volume (mm³) is calculated for a prolate ellipsoid from 2-dimensional tumor measurements: tumor volume (mm³)=(length×width2 [L×W²])÷2. Assuming unit density, tumor volume is converted to tumor weight (i.e., 1 mm³=1 mg).

The term “vascularization of a solid tumor” refers to the formation of blood vessels in a solid tumor. An agent that inhibits the vascularization of a tumor may inhibit vessel initiation, development, and/or maintenance leading to, for example, the reduction in the number and/or the density of vessels in a tumor.

The term “permeability of a solid tumor” refers to the permeability of a solid tumor to a therapeutic. A solid tumor may be said to be permeable to a therapeutic if the therapeutic is able to reach cells at the center of the tumor. An agent that increases the permeability of a tumor may for example, normalize, e.g., maintain, the vasculature of a solid tumor. Tumor vacularization and/or tumor permeability may be determined by a variety of methods known in the art, such as, e.g. by immunohistochemical analysis of biopsy specimens, or by imaging techniques, such as sonography of the tumor, computed tomography (CT) or magnetic resonance imaging (MRI) scans.

The term “% T/C” is the percentage of the mean tumor weight of the Treatment group (T) divided by the mean tumor weight of the Control group (C) multiplied by 100. A % T/C value of 42% or less is considered indicative of meaningful activity by the National Cancer Institute (USA).

The term “ ”% inhibition” is 100 minus the % T/C. A % inhibition value of 58% or more is considered indicative of meaningful activity by the National Cancer Institute (USA).

The term “statistically significant” or “statistical significance” refers to the likelihood that a result would have occurred by chance, given that an independent variable has no effect, or, that a presumed null hypothesis is true. Statistical significance can be determined by obtaining a “P-value” (P) which refers to the probability value. The p-value indicates how likely it is that the result obtained by the experiment is due to chance alone. In one embodiment of the invention, statistical significance can be determined by obtaining the p-value of the Two-Tailed One-Sample T-Test. A p-value of less than 0.05 is considered statistically significant, that is, not likely to be due to chance alone. Alternatively a statistically significant p-value may be between about 0.05 to about 0.04; between about 0.04 to about 0.03; between about 0.03 to about 0.02; between about 0.02 to about 0.01. In certain cases, the p-value may be less than 0.01. The p-value may be used to determine whether or not there is any statistically significant reduction in tumor size and/or vascularization of a solid tumor and/or any statistically significant increase in the permeability of a solid tumor when combination therapy is used to treat a subject having a tumor, e.g., a solid tumor. There is biological relevance to the P-value when statistical significance is observed over a series of treatment days rather than the occasional one day.

“Treating cancer” or “treating a subject having cancer” includes inhibition of the replication of cancer cells, inhibition of the spread of cancer, reduction in tumor size, lessening or reducing the number of cancerous cells in the body, and/or amelioration or alleviation of the symptoms of cancer. A treatment is considered therapeutic if there is a decrease in mortality and/or morbidity, and may be performed prophylactically, or therapeutically.

The term “immunotoxin” refers to a hybrid molecule formed by coupling an entire toxin or the A chain of a toxin to a binding molecule. The resulting molecule has the specificity of the binding molecule and has toxicity imparted by the toxin. Such toxins may be conjugated to an anti-LT-βR binding molecule or a biologic agent. Non-limiting examples of toxins include, e.g., maytansinoids, CC-1065 analogs, calicheamicin derivatives, anthracyclines, vinca alkaloids, ricin, diptheria toxin, and Pseudomonas exotoxin. Exemplary immunotoxic biologic agents include, but are not limited to an anti-CD33 antibody conjugated to calicheamicin, i.e., gemtuzumab ozogamicin, an anti-CD22 variable domain (Fv) fused to truncated Psuedomonas exotoxin, i.e., RFB4(dsFv)-PE38 (BL22), and an interleukin-2 (IL-2) fusion protein comprising diphtheria toxin, i.e., Denileukin diftitox.

A “patient” or “subject” or “host” refers to either a human or non-human animal.

The term “plant alkaloid” refers a compound belonging to a family of alkaline, nitrogen-containing molecules derived from plants that are biologically active and cytotoxic. Examples of plant alkoids include, but are not limited to, taxanes such as docetaxel and paclitaxel and vincas such as vinblastine, vincristine, and vinorelbine. In one embodiment, the plant alkaloid is Taxol.

### 2. Anti-Lymphotoxin-β-Receptor (LT-β-R) Binding Molecules

Preferred anti-LT-β-R binding molecules of the invention activate LT-β-R, i.e., are agonists of LT-β-R. U.S. Pat. No. 6,312,691 and WO 96/22788, the contents of which are hereby incorporated in their entirety, describe methods and compositions for the treatment of cancer using LT-β-R agonist, e.g., antibodies, to trigger cancer cell death. For example, U.S. Pat. No. 6,312,691 describes LT-β-R agonists for use in the invention including membrane-bound LT-α/β complexes, soluble LT-α/β complexes and anti-LT-β-R antibodies and methods for their preparation and purification.

In a preferred embodiment, the LT-β-R binding molecule is an anti-LT-β-R antibody. Various forms of anti-LT-β-R antibodies can be made using standard recombinant DNA techniques (Winter and Milstein, Nature, 349, pp. 293-99 (1991)).

In certain embodiments, the anti-LT-β-R binding molecule may be a polyclonal antibody. For example, antibodies may be raised in mammals by multiple subcutaneous or intraperitoneal injections of the relevant antigen and an adjuvant. This immunization typically elicits an immune response that comprises production of antigen-reactive antibodies from activated splenocytes or lymphocytes. The resulting antibodies may be harvested from the serum of the animal to provide polyclonal preparations.

In another embodiment, the anti-LT-β-R binding molecule is a monoclonal antibody. In certain embodiments, a monoclonal antibody of the invention may be selected from the group consisting of: BKA11, CDH10, BCG6, AGH1, BDA8, CBE11 and BHA10, each of which is described in WO 96/22788.

Monoclonal antibodies for use in the present invention may be produced in certain embodiments by a cell line selected from the group consisting of the cells lines in Table 1:

TABLE 1

| CELL LINE | mAb Name | ATCC Accession No. |
|---|---|---|
| a) AG.H1.5.1 | AGH1 | HB 11796 |
| b) BD.A8.AB9 | BDA8 | HB 11798 |
| c) BC.G6.AF5 | BCG6 | B 11794 |
| d) BH.A10 | BHA10 | B 11795 |
| e) BK.A11.AC10 | BKA11 | B 11799 |
| f) CB.E11.1 | CBE11 | B 11793 |
| g) CD.H10.1 | CDH10 | B 11797 |

The preparation of monoclonal antibodies is a well-known process (Kohler et al., Nature, 256:495 (1975)) in which the relatively short-lived, or mortal, lymphocytes from a mammal which has been injected with antigen are fused with an immortal tumor cell line (e.g. a myeloma cell line), thus, producing hybrid cells or “hybridomas” which are both immortal and capable of producing the genetically coded antibody of the B cell. The resulting hybrids are segregated into single genetic strains by selection, dilution, and regrowth with each individual strain comprising specific genes for the formation of a single antibody. They produce antibodies which are homogeneous against a desired antigen and, in reference to their pure genetic parentage, are termed “monoclonal.”

Hybridoma cells thus prepared are seeded and grown in a suitable culture medium that preferably contains one or more substances that inhibit the growth or survival of the unfused, parental myeloma cells. Those skilled in the art will appreciate that reagents, cell lines and media for the formation, selection and growth of hybridomas are commercially available from a number of sources and standardized protocols are well established. Generally, culture medium in which the hybridoma cells are growing is assayed for production of monoclonal antibodies against the desired antigen. Preferably, the binding specificity of the monoclonal antibodies produced by hybridoma cells is determined by immunoprecipitation or by an in vitro assay, such as a radioimmunoassay (RIA) or enzyme-linked immunoabsorbent assay (ELISA). After hybridoma cells are identified that produce antibodies of the desired specificity, affinity and/or activity, the clones may be subcloned by limiting dilution procedures and grown by standard methods (Goding, Monoclonal Antibodies: Principles and Practice, pp 59-103 (Academic Press, 1986)). It will further be appreciated that the monoclonal antibodies secreted by the subclones may be separated from culture medium, ascites fluid or serum by conventional purification procedures such as, for example, protein-A, hydroxylapatite chromatography, gel electrophoresis, dialysis or affinity chromatography.

In another embodiment, DNA encoding a desired monoclonal antibody may be readily isolated and sequenced using conventional procedures (e.g., by using oligonucleotide probes that are capable of binding specifically to genes encoding the heavy and light chains of murine antibodies). The isolated and subcloned hybridoma cells serve as a preferred source of such DNA. Once isolated, the DNA may be placed into expression vectors, which are then transfected into prokaryotic or eukaryotic host cells such as E. coli cells, simian COS cells, Chinese Hamster Ovary (CHO) cells or myeloma cells that do not otherwise produce immunoglobulins. More particularly, the isolated DNA (which may be modified as described herein) may be used to clone constant and variable region sequences for the manufacture antibodies as described in Newman et al., U.S. Pat. No. 5,658,570, filed Jan. 25, 1995, which is incorporated by reference herein. Essentially, this entails extraction of RNA from the selected cells, conversion to cDNA, and amplification by PCR using Ig specific primers. Suitable primers for this purpose are also described in U.S. Pat. No. 5,658,570. As will be discussed in more detail below, transformed cells expressing the desired antibody may be grown up in relatively large quantities to provide clinical and commercial supplies of the immunoglobulin.

Those skilled in the art will also appreciate that DNA encoding antibodies or antibody fragments may also be derived from antibody phage libraries, e.g., using pd phage or Fd phagemid technology. Exemplary methods are set forth, for example, in EP 368 684 B1; U.S. Pat. No. 5,969,108, Hoogenboom, H. R. and Chames. 2000. Immunol. Today 21:371; Nagy et al. 2002. Nat. Med. 8:801; Huie et al. 2001. Proc. Natl. Acad. Sci. USA 98:2682; Lui et al. 2002. J. Mol. Biol. 315:1063, each of which is incorporated herein by reference. Several publications (e.g., Marks et al. Bio/Technology 10:779-783 (1992)) have described the production of high affinity human antibodies by chain shuffling, as well as combinatorial infection and in vivo recombination as a strategy for constructing large phage libraries. In another embodiment, Ribosomal display can be used to replace bacteriophage as the display platform (see, e.g., Hanes et al. 2000. Nat. Biotechnol. 18:1287; Wilson et al. 2001. Proc. Natl. Acad. Sci. USA 98:3750; or Irving et al. 2001 J. Immunol. Methods 248:31.

In yet another embodiment, cell surface libraries can be screened for antibodies (Boder et al. 2000. Proc. Natl. Acad. Sci. USA 97:10701; Daugherty et al. 2000 J. Immunol. Methods 243:211. Such procedures provide alternatives to traditional hybridoma techniques for the isolation and subsequent cloning of monoclonal antibodies.

Yet other embodiments of the present invention comprise the generation of human or substantially human antibodies in nonhuman animals, such as transgenic animals harboring one or more human immunoglobulin transgenes. Such animals may be used as a source for splenocytes for producing hybridomas, as is described in U.S. Pat. No. 5,569,825, WO00076310, WO00058499 and WO00037504 and incorporated by reference herein.

Yet another highly efficient means for generating recombinant antibodies is disclosed by Newman, Biotechnology, 10: 1455-1460 (1992). Specifically, this technique results in the generation of primatized antibodies that contain monkey variable domains and human constant sequences. This reference is incorporated by reference in its entirety herein. Moreover, this technique is also described in commonly assigned U.S. Pat. Nos. 5,658,570, 5,693,780 and 5,756,096 each of which is incorporated herein by reference.

In another embodiment, lymphocytes can be selected by micromanipulation and the variable genes isolated. For example, peripheral blood mononuclear cells can be isolated from an immunized mammal and cultured for about 7 days in vitro. The cultures can be screened for specific IgGs that meet the screening criteria. Cells from positive wells can be isolated. Individual Ig-producing B cells can be isolated by FACS or by identifying them in a complement-mediated hemolytic plaque assay. Ig-producing B cells can be micromanipulated into a tube and the Vh and Vl genes can be amplified using, e.g., RT-PCR. The VH and VL genes can be cloned into an antibody expression vector and transfected into cells (e.g., eukaryotic or prokaryotic cells) for expression.

Alternatively, antibody-producing cell lines may be selected and cultured using techniques well known to the skilled artisan. Such techniques are described in a variety of laboratory manuals and primary publications. In this respect, techniques suitable for use in the invention as described below are described in Current Protocols in Immunology, Coligan et al., Eds., Green Publishing Associates and Wiley-Interscience, John Wiley and Sons, New York (1991) which is herein incorporated by reference in its entirety, including supplements.

Variable and constant region domains can be obtained from any source, (e.g., from one or more of the anti LT-β-R antibodies described herein) and be incorporated into a modified binding molecule of the invention. For example, to clone antibodies, mRNA can be isolated from hybridoma, spleen, or lymph cells, reverse transcribed into DNA, and antibody genes amplified by PCR. PCR may be initiated by consensus constant region primers or by more specific primers based on the published heavy and light chain DNA and amino acid sequences. As discussed above, PCR also may be used to isolate DNA clones encoding the antibody light and heavy chains. In this case the libraries may be screened by consensus primers or larger homologous probes, such as mouse constant region probes. Numerous primer sets suitable for amplification of antibody genes are known in the art (e.g., 5′ primers based on the N-terminal sequence of purified antibodies (Benhar and Pastan. 1994. Protein Engineering 7:1509); rapid amplification of cDNA ends (Ruberti, F. et al. 1994. J. Immunol. Methods 173:33); antibody leader sequences (Larrick et al. 1989 Biochem. Biophys. Res. Commun. 160:1250); or based on known variable region framework amino acid sequences from the Kabat (Kabat et al. 1991. Sequences of Proteins of Immunological Interest. Bethesda, Md.:JS Dep. Health Hum. Serv. 5^{th} ed.) or the V-base databases (e.g., Orlandi et al. 1989. Proc. Natl. Acad. Sci. USA 86:3833; Sblattero et al. 1998. Immunotechnology 3:271; or Krebber et al. 1997. J. Immunol. Methods 201:35). Constant region domains can be selected having a particular effector function (or lacking a particular effector function) or with a particular modification to reduce immunogenicity. Variable and constant domains can be cloned, e.g., using the polymerase chain reaction and primers which are selected to amplify the domain of interest. PCR amplification methods are described in detail in U.S. Pat. Nos. 4,683,195; 4,683,202; 4,800,159; 4,965,188; and in, e.g., “PCR Protocols: A Guide to Methods and Applications” Innis et al. eds., Academic Press, San Diego, Calif. (1990); Ho et al. 1989. Gene 77:51; Horton et al. 1993. Methods Enzymol. 217:270).

Alternatively, V domains can be obtained from libraries of V gene sequences from an animal of choice. Libraries expressing random combinations of domains, e.g., VH and VL domains, can be screened with a desired antigen to identify elements which have desired binding characteristics. Methods of such screening are well known in the art. For example, antibody gene repertoires can be cloned into a X bacteriophage expression vector (Huse, W D et al. 1989. Science 2476:1275). In addition, cells (Boder and Wittrup. 1997. Nat. Biotechnol. 15:553; Daugtherty, P. et al. 2000. J. Immunol. Methods. 243:211; Francisco et al. 1994. Proc. Natl. Acad. Sci. USA 90:10444; Georgiou et al. 1997. Nature Biotechnology 15:29) or viruses (e.g., Hoogenboom, H R. 1998 Immunotechnology 4:1 Winter et al. 1994. Annu. Rev. Immunol. 12:433; Griffiths, A D. 1998. Curr. Opin. Biotechnol. 9:102) expressing antibodies on their surface can be screened. Ribosomal display can also be used to screen antibody libraries (Hanes J., et al. 1998. Proc. Natl. Acad. Sci. USA 95:14130; Hanes, J. and Pluckthun. 1999. Curr. Top. Microbiol. Immunol. 243:107; He, M. and Taussig. 1997. Nucleic Acids Research 25:5132).

Preferred libraries for screening are human V gene libraries. VL and VH domains from a non-human source may also be used. In one embodiment, such non-human V domains can be altered to reduce their immunogenicity using art recognized techniques.

Libraries can be naïve, from immunized subjects, or semi-synthetic (Hoogenboom, H. R. and Winter. 1992. J. Mol. Biol. 227:381; Griffiths, A D, et al. EMBO J. 13:3245; de Kruif, J. et al. 1995. J. Mol. Biol. 248:97; Barbas, C. F., et al. 1992. Proc. Natl. Acad. Sci. USA 89:4457).

In addition, the sequences of many antibody V and C domains are known and such domains can be synthesized using methods well known in the art. In one embodiment, mutations can be made to immunoglobulin domains to create a library of nucleic acid molecules having greater heterogeneity (Thompson, J., et al. 1996. J. Mol. Biol. 256:77; Lamminmaki, U. et al. 1999. J. Mol. Biol. 291:589; Caldwell, R. C. and Joyce G F. 1992. PCR Methods Appl. 2:28; Caldwell R C and Joyce G F. 1994. PCR Methods Appl. 3:S136. Standard screening procedures can be used to select high affinity variants. In another embodiment, changes to VH and VL sequences can be made to increase antibody avidity, e.g., using information obtained from crystal structures using techniques known in the art.

Antigen recognition sites or entire variable regions may be derived from one or more parental antibodies. The parental antibodies can include naturally occurring antibodies or antibody fragments, antibodies or antibody fragments adapted from naturally occurring antibodies, antibodies constructed de novo using sequences of antibodies or antibody fragments known to be specific for the LT-beta receptor. Sequences that may be derived from parental antibodies include heavy and/or light chain variable regions and/or CDRs, framework regions or other portions thereof.

In one embodiment, the anti-LT-β-R binding molecule is a humanized antibody. To make humanized antibodies, animals are immunized with the desired antigen, the corresponding antibodies are isolated, and the portion of the variable region sequences responsible for specific antigen binding is removed. The animal-derived antigen binding regions are then cloned into the appropriate position of human antibody genes in which the antigen binding regions have been deleted. See, e.g. Jones, P. et al. (1986), Nature 321, 522-525 or Tempest et al. (1991) Biotechnology 9, 266-273. Also, transgenic mice, or other mammals, may be used to express humanized antibodies. Such humanization may be partial or complete. Humanized antibodies minimize the use of heterologous (inter-species) sequences in human antibodies, and are less likely to elicit immune responses in the treated subject. Humanized antibodies for use in the present invention may be produced in certain embodiments by a cell line selected from the group consisting of: E46.4 (huCBE11: ATCC patent deposit designation PTA-3357) or cell line E77.4 (huCBE11: ATCC patent deposit designation 3765).

In certain embodiments, the humanized antibody is humanized CBE11 (huCBE11) as described, including the nucleotide and amino acid sequence thereof, in PCT publication no. WO 02/30986 and U.S. application Ser. No. 10/412,406. In another embodiment, the humanized antibody is humanized BHA10 (huBHA10), as described, including the nucleotide and amino acid sequence thereof, in PCT publication no. WO/04002431 and U.S. Appln No. 11/021,819. Applicants' applications described above, the contents of which are hereby incorporated in their entirety, describe methods and compositions for the treatment of cancer using huCBE11 and huBHA10, to trigger cancer cell death.

In another embodiment, “chimeric” binding molecules can be constructed in which the antigen binding domain from an animal binding molecule is linked to a human constant domain (e.g. Cabilly et al., U.S. Pat. No. 4,816,567; Morrison et al., Proc. Natl. Acad. Sci. U.S.A., 81, pp. 6851-55 (1984)). Chimeric binding molecules reduce the observed immunogenic responses elicited by animal antibodies when used in human clinical treatments. Construction of different classes of recombinant anti-LT-β-R binding molecules can also be accomplished by making chimeric or humanized binding molecules comprising the anti-LT-β-R variable domains and human constant domains (CH1, CH2, CH3) isolated from different classes of immunoglobulins. For example, anti-LT-beta-R IgM binding molecules with increased antigen binding site valencies can be recombinantly produced by cloning the antigen binding site into vectors carrying the human mu. chain constant regions (Arulanandam et al., J. Exp. Med., 177, pp. 1439-50 (1993); Lane et al., Eur. J. Immunol., 22, pp. 2573-78 (1993); Traunecker et al., Nature, 339, pp. 68-70 (1989)). In addition, standard recombinant DNA techniques can be used to alter the binding affinities of recombinant binding molecules with their antigens by altering amino acid residues in the vicinity of the antigen binding sites. See, e.g. (Queen et al., Proc. Natl. Acad. Sci. U.S.A., 86, pp. 10029-33 (1989); WO 94/04679).

Anti-LT-β-R binding molecules of the invention may also be modified binding molecules. Exemplary modified binding molecules include, e.g., minibodies, diabodies, diabodies fused to CH3 molecules, tetravalent antibodies, intradiabodies (e.g., Jendreyko et al. 2003. J. Biol. Chem. 278:47813), bispecific antibodies, fusion proteins (e.g., antibody cytokine fusion proteins, proteins fused to at least a portion of an Fc receptor), bispecific antibodies. Other immunoglobulins (Ig) and certain variants thereof are described, for example in U.S. Pat. No. 4,745,055; EP 256,654; Faulkner et al., Nature 298:286 (1982); EP 120,694; EP 125,023; Morrison, J. Immun. 123:793 (1979); Kohler et al., Proc. Natl. Acad. Sci. USA 77:2197 (1980); Raso et al., Cancer Res. 41:2073 (1981); Morrison et al., Ann. Rev. Immunol. 2:239 (1984); Morrison, Science 229:1202 (1985); Morrison et al., Proc. Natl. Acad. Sci. USA 81:6851 (1984); EP 255,694; EP 266,663; and WO 88/03559. Reassorted immunoglobulin chains also are known. See, for example, U.S. Pat. No. 4,444,878; WO 88/03565; and EP 68,763 and references cited therein.

In one embodiment, an anti-LT-β-R binding molecule of the invention comprises an immunoglobulin heavy chain having deletion or substitution of at least one amino acid compared to wild type. For example, the mutation of one or more single amino acid in selected areas of the CH2 domain may be enough to substantially reduce Fc binding and thereby increase tumor localization. Similarly, it may be desirable to simply delete that part of one or more constant region domains that control the effector function (e.g. complement binding) to be modulated. Such partial deletions of the constant regions may improve selected characteristics of the antibody (serum half-life) while leaving other desirable functions associated with the subject constant region domain intact. Accordingly, in one embodiment, a binding molecule of the invention lacks all or part of a CH2 domain. Moreover, the constant regions of the anti-LT-β-R binding molecules of the invention may be modified through the mutation or substitution of one or more amino acids that enhances the profile of the resulting construct. In this respect it may be possible to disrupt the activity provided by a conserved binding site (e.g. Fc binding) while substantially maintaining the configuration and immunogenic profile of the modified binding molecule. Yet other preferred embodiments may comprise the addition of one or more amino acids to the constant region to enhance desirable characteristics such as effector function or provide for more cytotoxin or carbohydrate attachment. In such embodiments it may be desirable to insert or replicate specific sequences derived from selected constant region domains.

In another embodiment, mutations to naturally occurring hinge regions can be made. Such modifications to the constant region in accordance with the instant invention may easily be made using well known biochemical or molecular engineering techniques well within the skill of the art.

In one embodiment, an anti-LT-β-R binding molecule of the invention comprises modified constant regions wherein one or more domains are partially or entirely deleted (“domain deleted antibodies”). In especially preferred embodiments compatible modified binding molecules will comprise domain deleted constructs or variants wherein the entire CH2 domain has been removed.

In one embodiment, the modified binding molecules of the invention are minibodies. Minibodies are dimeric molecules made up of two polypeptide chains each comprising an ScFv molecule (a single polypeptide comprising one or more antigen binding sites, e.g., a VL domain linked by a flexible linker to a VH domain fused to a CH3 domain via a connecting peptide.

ScFv molecules can be constructed in a VH-linker-VL orientation or VL-linker-VH orientation.

The flexible hinge that links the VL and VH domains that make up the antigen binding site preferably comprises from about 10 to about 50 amino acid residues, see, e.g., Huston et al. 1988. Proc. Natl. Acad. Sci. USA 85:5879.

Methods of making single chain antibodies are well known in the art, e.g., Ho et al. 1989. Gene 77:51; Bird et al. 1988 Science 242:423; Pantoliano et al. 1991. Biochemistry 30:10117; Milenic et al. 1991. Cancer Research 51:6363; Takkinen et al. 1991. Protein Engineering 4:837.

Minibodies can be made by constructing an ScFv component and connecting peptide-CH3 component using methods described in the art (see, e.g., U.S. Pat. No. 5,837,821 or WO 94/09817A1). These components can be isolated from separate plasmids as restriction fragments and then ligated and recloned into an appropriate vector. Appropriate assembly can be verified by restriction digestion and DNA sequence analysis.

In another embodiment, a tetravalent minibody can be constructed. Tetravalent minibodies can be constructed in the same manner as minibodies, except that two ScFv molecules are linked using a flexible linker.

In another embodiment, the modified antibodies of the invention are CH2 domain deleted antibodies. Domain deleted constructs can be derived from a vector (e.g., from IDEC Pharmaceuticals, San Diego) encoding an IgG₁ human constant domain (see, e.g., WO 02/060955A2 and WO02/096948A2).

Besides the deletion of whole constant region domains, it will be appreciated that the antibodies of the present invention can be engineered to partially delete or substitute of a few amino acids or even a single amino acid. For example, the mutation of a single amino acid in selected areas of the C_{H}2 domain may be enough to substantially reduce Fc binding and thereby increase tumor localization. Similarly, it may be desirable to simply delete that part of one or more constant region domains that control the effector function (e.g. complement C1Q binding). Such partial deletions of the constant regions may improve selected characteristics of the antibody (serum half-life) while leaving other desirable functions associated with the subject constant region domain intact.

Creation of a C_{H}2 domain deleted version can be accomplished by way of overlapping PCR mutagenesis. The gamma 1 constant domain begins with a plasmid encoded Nhe I site with is in translational reading frame with the immunoglobulin sequence. A 5′ PCR primer was constructed encoding the Nhe I site as well as sequence immediately downstream. A 3′ PCR primer mate was constructed such that it anneals with the 3′ end to the immunoglobulin hinge region and encodes in frame the first several amino acids of the gamma 1 CH3 domain. A second PCR primer pair consisted of the reverse complement of the 3′ PCR primer from the first pair (above) as the 5′ primer and a 3′ primer that anneals at a loci spanning the BsrG I restriction site within the CH3 domain. Following each PCR amplification, the resultant products were utilized as template with the Nhe I and BsrG I 5′ and 3′, respectively primers. The amplified product was then cloned back into N5KG1 to create the plasmid N5KG1ΔC_{H}2. This construction places the intact CH3 domain immediately downstream and in frame with the intact hinge region. A similar procedure can be used to create a domain deleted construct in which the CH3 domain is immediately downstream of a connecting peptide. For example, a domain deleted version of the C2B8 antibody was created in this manner as described in U.S. Pat. Nos. 5,648,267 and 5,736,137 each of which is incorporated herein by reference.

In one embodiment, tetravalent domain-deleted antibodies can be produced by combining a DNA sequence encoding a domain deleted antibody with a ScFv molecule. For example, in one embodiment, these sequences are combined such that the ScFv molecule is linked at its N-terminus to the CH3 domain of the domain deleted antibody via a flexible linker.

In another embodiment a tetravalent antibody can be made by fusing an ScFv molecule to a connecting peptide, which is fused to a CH1 domain to construct an ScFv—Fab tetravalent molecule. (Coloma and Morrison. 1997. Nature Biotechnology. 15:159; WO 95/09917).

In another embodiment, the modified antibodies of the invention are diabodies. Diabodies are similar to scFv molecules, but usually have a short (less than 10 and preferably 1-5) amino acid residue linker connecting both V-domains, such that the VL and VH domains on the same polypeptide chain cannot interact. Instead, the VL and VH domain of one polypeptide chain interact with the VH and VL domain (respectively) on a second polypeptide chain (WO 02/02781). In one embodiment, a binding molecule of the invention is a diabody fused to at least one heavy chain portion. In a preferred embodiment, a binding molecule of the invention is a diabody fused to a CH3 domain.

In one embodiment a modified antibody of the invention comprises a tetravalent or bispecific tetravalent CH2 domain-deleted antibody with a scFv appended to the N-terminus of the light chain. In another embodiment of the invention, a binding molecule comprises a tetravalent or bispecific tetravalent CH2 domain-deleted antibody with a scFv appended to the N-terminus of the heavy chain. In one embodiment, the attachment of the scFv to the N-terminus results in reduced aggregation of the molecules as compared to molecules in which the scFv is attached at the carboxy-terminus. Other forms of modified binding molecules are also within the scope of the instant invention (e.g., WO 02/02781 A1; 5,959,083; 6,476,198 B1; US 2002/0103345 A1; WO 00/06605; Byrn et al. 1990. Nature. 344:667-70; Chamow and Ashkenazi. 1996. Trends Biotechnol. 14:52).

In still other embodiments, the anti-LT-β-R binding molecule is a multivalent anti-LT-β-R antibody. In one embodiment, a multivalent antibody comprises at least one antigen recognition site specific for a LT-β-R epitope. In certain embodiments, at least one of the antigen recognition sites is located within a scFv domain, while in other embodiments all antigen recognition sites are located within scFv domains.

Binding molecules may be bivalent, trivalent, tetravalent or pentavalent. In certain embodiments, the binding molecule is monospecific. In one embodiment, the binding molecule is specific for the epitope to which CBE11 binds. In other embodiments, the binding molecule of the invention is a monospecific tetravalent LT-β-R agonist antibody comprising four CBE11-antigen recognition sites. In another embodiment, the binding molecule is specific for the BHA10 epitope, and, in some embodiments, is tetravalent. In any of these embodiments, at least one antigen recognition site may be located on a scFv domain, and in certain of these embodiments, all antigen recognition sites may be located on scFv domains. Binding molecules may be multispecific, wherein the binding molecule of the invention binds to different epitopes on human LT-β receptors.

In certain embodiments, an anti-LT-β-R multivalent binding molecule may be multispecific, i.e., has at least one binding site that binds to LT-β-R or an epitope of LT-β-R and at least one second binding site that binds to a second, different molecule or to a second, different epitope of LT-β-R.

Multivalent, multispecific binding molecules may contain a heavy chain comprising two or more variable regions and/or a light chain comprising one or more variable regions wherein at least two of the variable regions recognize different epitopes on the LT-beta receptor.

In one embodiment, the multivalent binding molecule is an agonist of the lymphotoxin-beta receptor and comprises at least two domains that are capable of binding to the receptor and inducing LT-β-R signaling. These constructs can include a heavy chain containing two or more variable regions comprising antigen recognitions sites specific for binding the LT-beta receptor and a light chain containing one or more variable regions or can be constructed to comprise only heavy chains or light chains containing two or more variable regions comprising CDRs specific for binding the LT-beta receptor.

In certain embodiments of the invention, the binding molecule is specific for at least two members of the group of lymphotoxin-beta receptor (LT-β-R) epitopes consisting of the epitopes to which one of following antibodies bind: BKA11, CDH10, BCG6, AGH1, BDA8, CBE11 and BHA10. In one embodiment, the binding molecule is specific for the epitope to which the CBE11 and BHA10 antibodies bind, and in certain embodiments, is tetravalent. In one embodiment, the binding molecule has two CBE11-specific antigen recognition sites and two BHA10-specific recognition sites, wherein the binding molecule is a bispecific tetravalent LT-β-R agonist binding molecule. In any of the multispecific binding molecules, at least one antigen recognition site may be located on a scFv domain, and in certain embodiments, all antigen recognition sites are located on scFv domains.

In certain embodiments, the binding molecule is bispecific. Bispecific molecules can bind to two different target sites, e.g., on the same target molecule or on different target molecules. For example, in the case of antibodies, bispecific molecules can bind to two different epitopes, e.g., on the same antigen or on two different antigens. Bispecific molecules can also be used for human therapy, e.g., by directing cytotoxicity to a specific target (for example by binding to a pathogen or tumor cell and to a cytotoxic trigger molecule, such as the T cell receptor or the Fcγ receptor. Bispecific antibodies can also be used, e.g., as fibrinolytic agents or vaccine adjuvants.

In one embodiment, the bispecific binding molecules of the invention include those with at least one arm (ie. binding site) directed against LT-β-R and at least one arm directed against a cell-surface molecule or a soluble molecule. Exemplary cell-surface molecules include receptors or tumor cell antigens that are overexpressed on the surface of a tumor or neoplastic cell. Exemplary soluble molecules include anti-tumor agents (e.g., toxins, chemotherapeutics, and prodrugs thereof) and soluble enzymes (e.g. prodrug converting enzymes).

In one embodiment, the soluble molecule to which a bispecific binding molecule of the invention binds is a soluble ligand of the TNF family. Examples of TNF family ligands include, but are not limited to, LTA (which binds TNFR1/TNFRSF1A), TNF (which binds CD120b/TNFRSF1B), LTB (which binds LTBR/TNFRSF3), OX40L (which binds OX40/TNFRSF4), CD40L (which binds CD40/TNFRSF5), (which binds Fas/TNFRSF6 and DcR3/TNFRSF6B), CD27L (which binds CD27/TNFRSF7), CD30L (which binds CD30/TNFRSF8), 4-1-BB-L (which binds 4-1-BB/TNFRSF9), TRAIL (which binds TRAIL-R1/TNFRSF10A, TRAIL-R2/TNFRSF10B, TRAIL-R3/TNFRSF10C, and TRAIL-R4/TNFRSF10D), RANKL (which binds RANK/TNFRSF11A and Osteoprotegrin/TNFRSF11B), APO-3L (which binds APO-3/TNFRSF12 and DR3L/TNFRSF12L), APRIL (which binds TACI/TNFRSF13B), BAFF (which binds BAFFR/TNFRSF13A), LIGHT (which binds HVEM/TNFRSF14), NGF ligands (which bind LNGFR, e.g. NGF-β, NGF-2/NTF3, NTF5, BDNF, IFRD1), GITRL (which binds GITR/TNFRSF18), EDAR1 & XEDAR ligand, Fn14 ligand, and Troy/Trade ligand.

In another embodiment, the soluble molecule to which a bispecific binding molecule of the invention binds is a receptor of the TNF family, i.e., a TNF receptor other than LT-β-R. The limiting factor in the treatment of tumors with monospecific TNFR binding molecules is that often only a subset of tumors appears to be sensitive to such therapies. Bispecific TNFR binding molecules can specifically activate TNFRs, and enhance receptor signaling by, for example, bringing the TNFRs into close proximity which can thus target more than one TNFR or TNFR type and enhance signaling, thus providing an improved method of treating cancer. In one embodiment, the bispecific TNFR binding molecule increases the signal strength by binding to two or more TNFRs of the same type increasing the number of TNFRs being brought together. In another more preferred embodiment, the bispecific TNFR binding molecule is capable of binding to two different receptors of the TNF family.

In one embodiment, the TNFR to which a bispecific binding molecule binds contains a death domain. The term “death domain” refers to a cytoplasmic region of a TNF family receptor which is involved TNF-mediated cell death or apoptotic signaling and cell-cytotoxicity induction mediated by these receptors. This region couples the receptor to caspase activation via adaptor proteins resulting in activation of the extrinsic death pathway.

Examples of TNF receptors which contain death domains include, but are not limited to, TNFR1 (TNFRSF1A), Fas (TNFRSF6), DR-3 (TNFRSF6B), LNGFR (TNFRSF16) TRAIL-R1 (TNFRSF10A), TRAIL-R2 (TNFRSF10B) and DR6 (TNFRSF21). The apoptotic signaling of these receptors is modulated upon binding of a cognate ligand and formation of any of the following receptor-ligand pairs: TNFR1/TNFα, Fas/FasL, DR-3/DR-3LG, TRAIL-R1/TRAIL, or TRAIL-R2/TRAIL.

Bispecific binding molecules that target TNF family receptors containing death domains are useful for the treatment of cancer since the TNFRs of this type are often overexpressed on tumor cells and stimulating of the receptor can activate tumor cell apoptosis. In preferred embodiments, the death-domain containing TNFR to which the bispecific binding molecule of the invention binds is TRAIL-R2. TRAIL-R2 is preferred for human tumor therapy since its activation does not trigger hepatocyte apoptosis and hence should have reduced toxicity.

While the activation of some of death domain containing receptors, e.g. TNFR1 or Fas, has been toxic in in vivo applications, it is likely that tethering these receptors to other TNF receptors may diminish toxicity and thus render a toxic antibody less toxic.

A number of antibodies have been generated to death domain containing TNF receptors and are well known in the art. Such antibodies include anti-TNF-R1 monoclonal antibodies (R&D systems anti-TNF-R1; Tularik mAb #985, U.S. Pat. Nos. 6,110,690; 6,437,113), anti-Fas receptor mAb CH-11 (U.S. Pat. No. 6,312,691; WO 95/10540), anti-DR3 antibodies (U.S. Pat. No. 5,985,547; Johnson, et al. (1984) ImmunoBiology of HLA, ed. Dupont, B. O., Springer, New York; U.S. Pat. Nos. 6,462,176; 6,469,166), and anti-TRAIL-R antibodies (U.S. Pat. Nos. 5,763,223; 6,072,047; 6,284,236; 6,521,228; 6,569,642; 6,642,358; and U.S. Pat. No. 6,417,328).

Other target TNF family receptors with a role in tumor formation can be identified using existing RNA databases of receptor expression in various cell types which allow one to define TNF family receptors that are present or ideally overexpressed on various tumors. Moreover, existing RNA databases provide an additional advantage in that the pair of TNF family receptors to which a bispecific TNFR binding molecule of the invention binds could be optimized by identifying those receptor pairs that are more uniquely expressed on a tumor type or subset of tumors but are not abundant on normal tissues, especially liver and vasculature. In such a manner receptor pairs (or more) are identified that could deliver a potent signal to the tumor and spare normal tissues.

The multi specific binding molecules of the invention may be monovalent for each specificity or multivalent for each specificity. In one embodiment, a bispecific binding molecule of the invention may comprise one binding site that reacts with a first target molecule, i.e, LT-β-R, and one binding site that reacts with a second target molecule (e.g. a bispecific antibody molecule, fusion protein, or minibody). In another embodiment, a bispecific binding molecule of the invention may comprise two binding sites that react with a first target molecule, i.e, LT-β-R, and two binding sites that react with a second target molecule (e.g. a bispecific scFv2 tetravalent antibody, tetravalent minibody, or diabody).

In one embodiment, at least one binding site of a multispecific binding molecule of the invention is an antigen binding region of an anti-LT-β-R antibody, or an antigen binding fragment thereof.

In another embodiment, at least one binding site of multispecific binding molecule is a single chain Fv fragment. In one embodiment, the multispecific binding molecules of the invention are bivalent minibodies with one arm containing a scFv fragment directed to a first target molecule, i.e, LT-β-R, and a second arm containing a scFv directed to a second target molecule.

In another embodiment, the multispecific binding molecules of the invention are scFv tetravalent minibodies, with each heavy chain portion of the scFv tetravalent minibody containing first and second scFv fragments. Said second scFv fragment may be linked to the N-terminus of the first scFv fragment (e.g. bispecific N_{H} scFv tetravalent minibodies or bispecific N_{L} scFv tetravalent minibodies). Alternatively, the second scFv fragment may be linked to the C-terminus of said heavy chain portion containing said first scFv fragment (e.g. bispecific C-scFv tetravalent minibodies). In one embodiment, the first and second scFv fragments of may bind the same or different target molecule. Where the first and second scFv fragments of a first heavy chain portion of a bispecific tetravalent minibody bind the same target molecule, at least one of the first and second scFv fragments of the second heavy chain portion of the bispecific tetravalent minibody binds a different target molecule.

In another embodiment, the multispecific binding molecules of the invention are bispecific diabodies, with each arm of the diabody comprising tandem scFv fragments. In one embodiment, a bispecific diabody may comprise a first arm with a first binding specificity and a second arm with a second binding specificity. In another embodiment, each arm of the diabody may comprise a first scFv fragment with a first binding specificity and a second scFv fragment with a second binding specificity.

In another embodiment, the multispecific binding molecules of the invention are scFv2 tetravalent antibodies with each heavy chain portion of the scFv2 tetravalent antibody containing a scFv fragment. The scFv fragments may be linked to the N-termini of a variable region of the heavy chain portions (e.g. bispecific N_{H} scFv2 tetravalent antibodies or bispecific N_{L} scFv2 tetravalent antibodies). Alternatively, the scFv fragments may be linked to the C-termini of the heavy chain portions of the scFv2 tetravalent antibody (e.g. bispecific C-scFv2 tetravalent antibodies. Each heavy chain portion of the scFv2 tetravalent antibody may have variable regions and scFv fragments that bind the same or different target molecules. Where the scFv fragment and variable region of a first heavy chain portion of a bispecific scFc2 tetravalent antibody bind the same target molecule, at least one of the first and second scFv fragments of the second heavy chain portion of the bispecific tetravalent minibody binds a different target molecule.

In another embodiment, the multispecific binding molecules of the invention are scFv2 tetravalent domain-deleted antibodies with each heavy chain portion of the scFv2 tetravalent antibody containing a scFv fragment. The scFv fragments may be linked to the N-termini of a variable region of the heavy chain portions (e.g. bispecific N_{H} scFv2 tetravalent domain-deleted antibodies or bispecific N_{L} scFv2 tetravalent antibodies. Alternatively, the scFv fragments may be linked to the C-termini of the heavy chain portions of the scFv2 tetravalent antibody (e.g. bispecific C-scFv2 tetravalent antibodies).

Methods for making multivalent multispecific antibodies are known in the art. Traditional production of full length bispecific antibodies is based on the coexpression of two immunoglobulin heavy chain-light chain pairs, where the two chains have different specificities (Milstein et al., Nature, 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J., 10:3655-3659 (1991).

Multivalent, anti-LT-β-R antibodies may be constructed in a variety different ways using a variety of different sequences derived from parental anti-LT-β-R antibodies, including murine or humanized BHA10 (Browning et al., J. Immunol. 154: 33 (1995); Browning et al. J. Exp. Med. 183:867 (1996)) and/or murine or humanized CBE11 (U.S. Pat. No. 6,312,691).

Methods of producing bispecific molecules are well known in the art. For example, recombinant technology can be used to produce bispecific molecules, e.g., diabodies, single-chain diabodies, tandem scFvs, etc. Exemplary techniques for producing bispecific molecules are known in the art (e.g., Kontermann et al. Methods in Molecular Biology Vol. 248: Antibody Engineering: Methods and Protocols. Pp 227-242 US 2003/0207346 A1 and the references cited therein). In one embodiment, a multimeric bispecific molecules are prepared using methods such as those described e.g., in US 2003/0207346 A1 or U.S. Pat. No. 5,821,333, or US2004/0058400.

In another embodiment, a multispecific binding molecule of the invention is a multispecific fusion protein. As used herein the phrase “multispecific fusion protein” designates fusion proteins having at least two binding specificities (i.e. combining two or more binding domains. Multispecific fusion proteins can be assembled as heterodimers, heterotrimers or heterotetramers, essentially as disclosed in WO 89/02922 (published Apr. 6, 1989), in EP 314, 317 (published May 3, 1989), and in U.S. Pat. No. 5,116,964 issued May 2, 1992. Preferred multispecific fusion proteins are bispecific.

In one embodiment, the subject bispecific molecule is expressed in an expression system used to express antibody molecules, for example mammalian cells, yeast such as Picchia, E. coli, Bacculovirus, etc. In one embodiment, the subject bispecific molecule is expressed in the NEOSPLA vector system (see, e.g., U.S. Pat. No. 6,159,730). This vector contains the cytomegalovirus promoter/enhancer, the mouse beta globin major promoter, the SV40 origin of replication, the bovine growth hormone polyadenylation sequence, neomycin phosphotransferase exon 1 and exon 2, the dihydrofolate reductase gene and leader sequence.

A variety of other multivalent antibody constructs may be developed by one of skill in the art using routine recombinant DNA techniques, for example as described in PCT International Application No. PCT/US86/02269; European Patent Application No. 184,187; European Patent Application No. 171,496; European Patent Application No. 173,494; PCT International Publication No. WO 86/01533; U.S. Pat. No. 4,816,567; European Patent Application No. 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Cancer Res. 47:999-1005; Wood et al. (1985) Nature 314:446-449; Shaw et al. (1988) J. Natl. Cancer Inst. 80:1553-1559); Morrison (1985) Science 229:1202-1207; Oi et al. (1986) BioTechniques 4:214; U.S. Pat. No. 5,225,539; Jones et al. (1986) Nature 321:552-525; Verhoeyan et al. (1988) Science 239:1534; Beidler et al. (1988) J. Immunol. 141:4053-4060; and Winter and Milstein, Nature, 349, pp. 293-99 (1991)). Preferably non-human antibodies are “humanized” by linking the non-human antigen binding domain with a human constant domain (e.g. Cabilly et al., U.S. Pat. No. 4,816,567; Morrison et al., Proc. Natl. Acad. Sci. U.S.A., 81, pp. 6851-55 (1984)).

Other methods which may be used to prepare multivalent antibody constructs are described in the following publications: Ghetie, Maria-Ana et al. (2001) Blood 97:1392-1398; Wolff, Edith A. et al. (1993) Cancer Research 53:2560-2565; Ghetie, Maria-Ana et al. (1997) Proc. Natl. Acad. Sci. 94:7509-7514; Kim, J. C. et al. (2002) Int. J. Cancer 97(4):542-547; Todorovska, Aneta et al. (2001) Journal of Immunological Methods 248:47-66; Coloma M. J. et al. (1997) Nature Biotechnology 15:159-163; Zuo, Zhuang et al. (2000) Protein Engineering (Suppl.) 13(5):361-367; Santos A. D., et al. (1999) Clinical Cancer Research 5:3118s-3123s; Presta, Leonard G. (2002) Current Pharmaceutical Biotechnology 3:237-256; van Spriel, Annemiek et al., (2000) Review Immunology Today 21(8) 391-397.

In some embodiments, the binding molecules and binding molecule fragments of the invention may be chemically modified to provide a desired effect. For example, pegylation of antibodies and antibody fragments of the invention may be carried out by any of the pegylation reactions known in the art, as described, for example, in the following references: Focus on Growth Factors 3:4-10 (1992); EP 0 154 316; and EP 0 401 384 (each of which is incorporated by reference herein in its entirety). Preferably, the pegylation is carried out via an acylation reaction or an alkylation reaction with a reactive polyethylene glycol molecule (or an analogous reactive water-soluble polymer). A preferred water-soluble polymer for pegylation of the binding molecules and binding molecule fragments of the invention is polyethylene glycol (PEG). As used herein, “polyethylene glycol” is meant to encompass any of the forms of PEG that have been used to derivatize other proteins, such as mono (Cl—ClO) alkoxy- or aryloxy-polyethylene glycol.

Methods for preparing pegylated binding molecules and binding molecule fragments of the invention will generally comprise the steps of (a) reacting the binding molecule or binding molecule fragment with polyethylene glycol, such as a reactive ester or aldehyde derivative of PEG, under conditions whereby the binding molecule or binding molecule fragment becomes attached to one or more PEG groups, and (b) obtaining the reaction products. It will be apparent to one of ordinary skill in the art to select the optimal reaction conditions or the acylation reactions based on known parameters and the desired result.

Pegylated binding molecules and binding molecule fragments may generally be used to treat conditions that may be alleviated or modulated by administration of the binding molecules and binding molecule fragments described herein. Generally the pegylated binding molecules and binding molecule fragments have increased half-life, as compared to the nonpegylated binding molecules and binding molecule fragments. The pegylated binding molecules and binding molecule fragments may be employed alone, together, or in combination with other pharmaceutical compositions.

In other embodiments of the invention the binding molecules or antigen-binding fragments thereof are conjugated to albumen using art recognized techniques.

In another embodiment of the invention, binding molecules, or fragments thereof, are modified to reduce or eliminate potential glycosylation sites. Such modified antibodies are often referred to as “aglycosylated” binding molecules. In order to improve the binding affinity of a binding molecule or antigen-binding fragment thereof, glycosylation sites of the binding molecule can be altered, for example, by mutagenesis (e.g., site-directed mutagenesis). “Glycosylation sites” refer to amino acid residues which are recognized by a eukaryotic cell as locations for the attachment of sugar residues. The amino acids where carbohydrate, such as oligosaccharide, is attached are typically asparagine (N-linkage), serine (O-linkage), and threonine (O-linkage) residues. In order to identify potential glycosylation sites within an binding molecule or antigen-binding fragment, the sequence of the binding molecule is examined, for example, by using publicly available databases such as the website provided by the Center for Biological Sequence Analysis (see http://www.cbs.dtu.dk/services/NetNGlyc/for predicting N-linked glycoslyation sites) and http://www.cbs.dtu.dk/services/NetOGlyc/for predicting O-linked glycoslyation sites). Additional methods for altering glycosylation sites of binding molecules are described in U.S. Pat. Nos. 6,350,861 and 5,714,350.

In yet another embodiment of the invention, binding molecules or antigen binding fragments thereof can be altered wherein the constant region of the binding molecule is modified to reduce at least one constant region-mediated biological effector function relative to an unmodified binding molecule. To modify a binding molecule of the invention such that it exhibits reduced binding to the Fc receptor (FcR), the immunoglobulin constant region segment of the binding molecule can be mutated at particular regions necessary for FcR interactions (see e.g., Canfield et al (1991) J. Exp. Med. 173:1483; and Lund, J. et al. (1991) J. of Immunol. 147:2657). Reduction in FcR binding ability of the binding molecule may also reduce other effector functions which rely on FcR interactions, such as opsonization and phagocytosis and antigen-dependent cellular cytotoxicity.

In a particular embodiment the invention further features binding molecules having altered effector function, such as the ability to bind effector molecules, for example, complement or a receptor on an effector cell. In particular, the humanized binding molecules of the invention have an altered constant region, e.g., Fc region, wherein at least one amino acid residue in the Fc region has been replaced with a different residue or side chain thereby reducing the ability of the binding molecule to bind the FcR. Reduction in FcR binding ability of the binding molecule may also reduce other effector functions which rely on FcR interactions, such as opsonization and phagocytosis and antigen-dependent cellular cytotoxicity. In one embodiment, the modified humanized binding molecule is of the IgG class, comprises at least one amino acid residue replacement in the Fc region such that the humanized binding molecule has an altered effector function, e.g., as compared with an unmodified humanized binding molecule. In particular embodiments, the humanized binding molecule of the invention has an altered effector function such that it is less immunogenic (e.g., does not provoke undesired effector cell activity, lysis, or complement binding), and/or has a more desirable half-life while retaining specificity for LTβR or a ligand thereof.

Alternatively, the invention features humanized binding molecules having altered constant regions to enhance FcR binding, e.g., FcγR3 binding. Such binding molecules are useful for modulating effector cell function, e.g., for increasing ADCC activity, e.g., particularly for use in oncology applications of the invention.

As used herein, “antibody-dependent cell-mediated cytotoxicity” and “ADCC” refer to a cell-mediated reaction in which nonspecific cytotoxic cells that express FcRs (e.g. Natural Killer (NK) cells, neutrophils, and macrophages) recognize bound binding molecule on a target cell and subsequently cause lysis of the target cell. The primary cells for mediating ADCC, NK cells, express FcγRIII only, whereas monocytes express FcγRI, FcγRII and FcγRIII. of the antibody, e.g., a conjugate of the binding molecule and another agent or binding molecule.

In still another embodiment, the anti-LT-β-R binding molecules or biologic agents of the invention can be conjugated to a chemotherapeutic agent or a toxin for use in the methods of the invention. Exemplary chemotherapeutics that can be conjugated to the antibodies of the present invention include, but are not limited to radioconjugates (90Y, 131I, 99 mTc, 111In, 186Rh, et al.).

The cytotoxic effects of LT-β-R binding molecules on a tumor may be enhanced by the presence of a LT-β-R activating agent, particularly IFN-gamma. Any agent which is capable of inducing interferons, preferably IFN-gamma, and which potentiates the cytotoxic effects of LT-alpha/beta heteromeric complexes and anti-LT-β-R binding molecules on tumor cells falls within the group of LT-β-R binding molecules. For example, clinical experiments have demonstrated interferon induction by double stranded RNA (dsRNA) treatment. Accordingly, polyriboguanylic/polyribocytidylic acid (poly-rG/rC) and other forms of dsRNA are effective as interferon inducers (Juraskova et al., Eur. J. Pharmacol 221, pp. 107-11 (1992)).

The LT-β-R binding molecules produced as described above may be purified to a suitable purity for use as a pharmaceutical composition. Generally, a purified composition will have one species that comprises more than about 85 percent of all species present in the composition, more than about 85%, 90%, 95%, 99% or more of all species present. The object species may be purified to essential homogeneity (contaminant species cannot be detected in the composition by conventional detection methods) wherein the composition consists essentially of a single species. A skilled artisan may purify a polypeptide of the invention using standard techniques for protein purification in light of the teachings herein. Purity of a polypeptide may be determined by a number of methods known to those of skill in the art, including for example, amino-terminal amino acid sequence analysis, gel electrophoresis and mass-spectrometry analysis.

### 3. Biologic Agents

Biological agents (also called biologics) are the product of a biological system, e.g., an organism, cell, or recombinant system. Examples of such biologic agents include nucleic acid molecules, e.g., antisense nucleic acid molecules, interferons, interleukins, colony-stimulating factors, antibodies, e.g., monoclonal antibodies, and cytokines. Exemplary biologic agents are discussed in more detail below.

Interferons (IFN) are a type biologic agent that naturally occurs in the body. Interferons are also produced in the laboratory and given to cancer patients in biological therapy. They have been shown to improve the way a cancer patient's immune system acts against cancer cells. Interferons may work directly on cancer cells to slow their growth, or they may cause cancer cells to change into cells with more normal behavior. Some interferons may also stimulate natural killer cells (NK) cells, T cells, and macrophages—types of white blood cells in the bloodstream that help to fight cancer cells.

Interleukins (IL) stimulate the growth and activity of many immune cells. They are proteins (cytokines and chemokines) that occur naturally in the body, but can also be made in the laboratory. Some interleukins stimulate the growth and activity of immune cells, such as lymphocytes, which work to destroy cancer cells.

Colony-stimulating factors (CSFs) are proteins given to patients to encourage stem cells within the bone marrow to produce more blood cells. The body constantly needs new white blood cells, red blood cells, and platelets, especially when cancer is present. CSFs are given, along with chemotherapy, to help boost the immune system. When cancer patients receive chemotherapy, the bone marrow's ability to produce new blood cells is suppressed, making patients more prone to developing infections. Parts of the immune system cannot function without blood cells, thus colony-stimulating factors encourage the bone marrow stem cells to produce white blood cells, platelets, and red blood cells. With proper cell production, other cancer treatments can continue enabling patients to safely receive higher doses of chemotherapy.

Antibodies, e.g., monoclonal antibodies, are agents, produced in the laboratory, that bind to cancer cells. When cancer-destroying agents are introduced into the body, they seek out the antibodies and kill the cancer cells. Monoclonal antibody agents do not destroy healthy cells. Monoclonal antibodies achieve their therapeutic effect through various mechanisms. They can have direct effects in producing apoptosis or programmed cell death. They can block growth factor receptors, effectively arresting proliferation of tumor cells. In cells that express monoclonal antibodies, they can bring about anti-idiotype antibody formation.

Examples of antibodies which may be used in the combination treatment of the invention include anti-CD20 antibodies, such as, but not limited to, cetuximab, Tositumomab, rituximab, and Ibritumomab. Anti-HER2 antibodies may also be used in combination with an anti-LT-β-R antibody for the treatment of cancer. In one embodiment, the anti-HER2 antibody is Trastuzumab (Herceptin). Other examples of antibodies which may be used in combination with an anti-LT-β-R antibody for the treatment of cancer include anti-CD52 antibodies (e.g., Alelmtuzumab), anti-CD-22 antibodies (e.g., Epratuzumab), and anti-CD33 antibodies (e.g., Gemtuzumab ozogamicin). In certain embodiments, the biologic agent is an antibody that inhibits angiogenesis is an anti-VEGF antibody, e.g., bevacizumab. In other embodiments, the biologic agent is an antibody which is an anti-EGFR antibody e.g., cetuximab. Another example is the anti-glycoprotein 17-1A antibody edrecolomab.

Cytokine therapy uses proteins (cytokines) to help a subject's immune system recognize and destroy those cells that are cancerous. Cytokines are produced naturally in the body by the immune system, but can also be produced in the laboratory. This therapy is used with advanced melanoma and with adjuvant therapy (therapy given after or in addition to the primary cancer treatment). Cytokine therapy reaches all parts of the body to kill cancer cells and prevent tumors from growing.

Fusion proteins may also be used. For example, recombinant human Apo2L/TRAIL (Genentech) may be used in a combination therapy. Apo2/TRAIL is the first dual pro-apoptotic receptor agonist designed to activate both pro-apoptotic receptors DR4 and DR5, which are involved in the regulation of apoptosis (programmed cell death).

Antisense nucleic acid molecules may also be used in the methods of the invention. As used herein, an “antisense” nucleic acid comprises a nucleotide sequence which is complementary to a “sense” nucleic acid encoding a protein, e.g., complementary to the coding strand of a double-stranded cDNA molecule, complementary to an mRNA sequence or complementary to the coding strand of a gene. Accordingly, an antisense nucleic acid can hydrogen bond to a sense nucleic acid.

In one embodiment, a biologic agent is an siRNA molecule, e.g., of a molecule that enhances angiogenesis, e.g., bFGF, VEGF and EGFR. In one embodiment, a biologic agent that inhibits angiogenesis mediates RNAi. RNA interference (RNAi) is a post-transcriptional, targeted gene-silencing technique that uses double-stranded RNA (dsRNA) to degrade messenger RNA (mRNA) containing the same sequence as the dsRNA (Sharp, P. A. and Zamore, P. D. 287, 2431-2432 (2000); Zamore, P. D., et al. Cell 101, 25-33 (2000). Tuschl, T. et al. Genes Dev. 13, 3191-3197 (1999); Cottrell T R, and Doering T L. 2003. Trends Microbiol. 11:37-43; Bushman F.2003. Mol. Therapy. 7:9-10; McManus M T and Sharp P A. 2002. Nat Rev Genet. 3:737-47). The process occurs when an endogenous ribonuclease cleaves the longer dsRNA into shorter, e.g., 21- or 22-nucleotide-long RNAs, termed small interfering RNAs or siRNAs. The smaller RNA segments then mediate the degradation of the target mRNA. Kits for synthesis of RNAi are commercially available from, e.g. New England Biolabs or Ambion. In one embodiment one or more of the chemistries described herein for use in antisense RNA can be employed in molecules that mediate RNAi.

The use of antisense nucleic acids to downregulate the expression of a particular protein in a cell is well known in the art (see e.g., Weintraub, H. et al., Antisense RNA as a molecular tool for genetic analysis, Reviews—Trends in Genetics, Vol. 1(1) 1986; Askari, F. K. and McDonnell, W. M. (1996) N. Eng. J. Med. 334:316-318; Bennett, M. R. and Schwartz, S. M. (1995) Circulation 92:1981-1993; Mercola, D. and Cohen, J. S. (1995) Cancer Gene Ther. 2:47-59; Rossi, J. J. (1995) Br. Med. Bull. 51:217-225; Wagner, R. W. (1994) Nature 372:333-335). An antisense nucleic acid molecule comprises a nucleotide sequence that is complementary to the coding strand of another nucleic acid molecule (e.g., an mRNA sequence) and accordingly is capable of hydrogen bonding to the coding strand of the other nucleic acid molecule. Antisense sequences complementary to a sequence of an mRNA can be complementary to a sequence found in the coding region of the mRNA, the 5′ or 3′ untranslated region of the mRNA or a region bridging the coding region and an untranslated region (e.g., at the junction of the 5′ untranslated region and the coding region). Furthermore, an antisense nucleic acid can be complementary in sequence to a regulatory region of the gene encoding the mRNA, for instance a transcription initiation sequence or regulatory element. Preferably, an antisense nucleic acid is designed so as to be complementary to a region preceding or spanning the initiation codon on the coding strand or in the 3′ untranslated region of an mRNA.

Given the coding strand sequences of a molecule that enhances angiogenesis, antisense nucleic acids of the invention can be designed according to the rules of Watson and Crick base pairing. The antisense nucleic acid molecule can be complementary to the entire coding region of the mRNA, but more preferably is an oligonucleotide which is antisense to only a portion of the coding or noncoding region of the mRNA. For example, the antisense oligonucleotide can be complementary to the region surrounding the translation start site of the mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. To inhibit expression in cells, one or more antisense oligonucleotides can be used. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In another embodiment, an antisense nucleic acid of the invention is a compound that mediates RNAi. RNA interfering agents include, but are not limited to, nucleic acid molecules including RNA molecules which are homologous to the target gene or genomic sequence, “short interfering RNA” (siRNA), “short hairpin” or “small hairpin RNA” (shRNA), and small molecules which interfere with or inhibit expression of a target gene by RNA interference (RNAi). RNA interference is a post-transcriptional, targeted gene-silencing technique that uses double-stranded RNA (dsRNA) to degrade messenger RNA (mRNA) containing the same sequence as the dsRNA (Sharp, P. A. and Zamore, P. D. 287, 2431-2432 (2000); Zamore, P. D., et al. Cell 101, 25-33 (2000). Tuschl, T. et al. Genes Dev. 13, 3191-3197 (1999)). The process occurs when an endogenous ribonuclease cleaves the longer dsRNA into shorter, 21- or 22-nucleotide-long RNAs, termed small interfering RNAs or siRNAs. The smaller RNA segments then mediate the degradation of the target mRNA. Kits for synthesis of RNAi are commercially available from, e.g. New England Biolabs and Ambion. In one embodiment one or more of the chemistries described above for use in antisense RNA can be employed.

Nucleic acid molecules encoding molecules that inhibit angiogenesis may be introduced into the subject in a form suitable for expression of the encoded protein in the cells of the subject may also be used in the methods of the invention. Exemplary molecules that inhibit angiogenesis include, but are not limited to, TSP-1, TSP-2, IFN-α, IFN-β, angiostatin, endostsin, tumastatin, canstatin, VEGI, PEDF, vasohibin, and the 16 kDa fragment of prolactin 2-Methoxyestradiol (see, Kerbel (2004) J. Clin Invest 114:884, for review).

For example, a full length or partial cDNA sequence is cloned into a recombinant expression vector and the vector is transfected into a cell using standard molecular biology techniques. The cDNA can be obtained, for example, by amplification using the polymerase chain reaction (PCR) or by screening an appropriate cDNA library. The nucleotide sequences of the cDNA can be used for the design of PCR primers that allow for amplification of a cDNA by standard PCR methods or for the design of a hybridization probe that can be used to screen a cDNA library using standard hybridization methods. Following isolation or amplification of the cDNA, the DNA fragment is introduced into a suitable expression vector.

It should be noted that more than one biologic agent may be administered in combination with an anti-LT-β-R binding molecule.

Thus, the invention provides for the use of a combination therapy and at least one additional agent to treat cancer, i.e., reduce tumor size and/or tumor vascularization and/or increase tumor permeability.

The present invention also includes a method of treating cancer by sensitizing tumor cells with an anti-LT-β-R binding molecule, such that, e.g., the vasculature of a solid tumor is increased by, e.g., increasing the permeability, e.g., normalizing, e.g., maintaining, the vasculature, and then subsequently administering a at least one additional agent. In one embodiment, a chemotherapeutic agent is administered in addition to the combination therapy.

In preferred embodiments, the second agent inhibits angiogenesis. In certain preferred embodiments, the agent that inhibits angiogenesis is a biologic agent. The biologic agent that inhibits angiogenesis may be an antibody or antigen binding fragment thereof. In certain embodiments, the biologic agent that inhibits angiogenesis is an anti-VEGF antibody, e.g., bevacizumab. In other embodiments, the biologic agent is an anti-EGFR antibody e.g., cetuximab.

In one embodiment of the invention the at least one biologic agent is selected from the group consisting of rituximab, trastuzumab, tositumomab, ibritumomab, alelmtuzumab, epratuzumab, gemtuzumab ozogamicin, oblimersen, and panitumumab.

In another embodiment, the agent that inhibits angiogenesis is a small molecule. In one embodiment, the small molecule is an epidermal growth factor type 1/epidermal growth factor receptor (HER1/EGFR) inhibitor, e.g., erlotinib.

In another embodiment of the invention, the biologic agent is an interferon or an interleukin.

Various forms of the biologic agents may be used. These include, without limitation, such forms as proform molecules, uncharged molecules, molecular complexes, salts, ethers, esters, amides, and the like, which are biologically activated when implanted, injected or otherwise inserted into the tumor.

### 4. Therapeutic Methods

The present invention further provides novel therapeutic methods of reducing tumor size in a subject having a tumor of a size greater than about 2 mm×2 mm, decreasing vascularization of a solid tumor, e.g., a tumor of a size greater than about 2 mm×2 mm, in a subject having a solid tumor, and/or increasing permeability of a solid tumor, e.g., a tumor of a size greater than about 2 mm×2 mm, in a subject having a solid tumor. The methods generally involve administering to the subject a combination therapy. In certain embodiments of the invention, the methods may further comprise administering to the subject a chemotherapeutic agent.

The methods of the present invention may be used to treat cancers, including but not limited to treating solid tumors, e.g., a carcinoma. Examples of solid tumors, e.g., carcinomas, that can be treated by compounds of the present invention, include but are not limited to breast, testicular, lung, ovary, uterine, cervical, pancreatic, non small cell lung (NSCLC), colon, as well as prostate, gastric, skin, stomach, esophagus and bladder cancer. In one embodiment, the tumor is a colon tumor. In another embodiment, the tumor is selected from the group consisting of a colon tumor, a cervical tumor, a gastric tumor, or a pancreatic tumor. In another embodiment, the tumor is selected from the group consisting of Stage I, Stage II, Stage III, and Stage IV tumors.

In one embodiment of the invention, the subject combination therapies are used to treat established tumors, e.g., tumors of sufficient size such that nutrients can no longer permeate to the center of the tumor from the subject's vasculature by osmosis and therefore the tumor requires its own vascular supply to receive nutrients, i.e, a vascularized tumor. In one embodiment, a combination therapy is used to treat a tumor having dimensions of at least about 1 mm×1 mm. In another embodiment of the invention, a combination therapy is used to treat a tumor that is at least about 2 mm×2 mm. In yet another embodiment of the invention, a combination therapy is used to treat a tumor that is at least about 5 mm×5 mm. In other embodiments of the invention the tumor has a volume of at least about 1 cm³. In one embodiment, a combination therapy of the invention is used to treat a tumor that is large enough to be found by palpation or by imaging techniques well known in the art, such as MRI, ultrasound, or CAT scan.

In certain embodiments of the invention, the subject methods result in a % tumor inhibition of greater than about 58%, 60%, 65%, 70%, 75%, 80%, 85%, 90%, 95%, 100%. In one embodiment, the administration of an anti-lymphotoxin-beta receptor (LT-β-R) binding molecule, or an antigen-binding fragment thereof, and at least one agent that inhibits angiogenesis results in a % tumor inhibition of about 58% or greater.

In certain embodiments, the method comprises parenterally administering an effective amount of an anti-LT-β-R binding molecule and a second agent to a subject. In one embodiment, the method comprises intraarterial administration of an anti-LT-β-R binding molecule and at least one additional agent to a subject. In other embodiments, the method comprises administering an effective amount of an anti-LT-β-R binding molecule and at least one additional agent directly to the arterial blood supply of a tumor in a subject. In one embodiment, the methods comprise administering an effective amount of an anti-LT-β-R binding molecule and at least one additional agent directly to the arterial blood supply of the cancerous tumor using a catheter. In embodiments where a catheter is used to administer an anti-LT-β-R binding molecule and at least one additional agent, the insertion of the catheter may be guided or observed by fluoroscopy or other method known in the art by which catheter insertion may be observed and/or guided. In another embodiment, the method comprises chemoembolization. For example a chemoembolization method may comprise blocking a vessel feeding the cancerous tumor with a composition comprised of a resin-like material mixed with an oil base (e.g., polyvinyl alcohol in Ethiodol) and one or more biologic agents. In still other embodiments, the method comprises systemic administration of an anti-LT-β-R binding molecule and at least one additional agent to a subject.

In general, chemoembolization or direct intraarterial or intravenous injection therapy utilizing pharmaceutical compositions of the present invention is typically performed in a similar manner, regardless of the site. Briefly, angiography (a road map of the blood vessels), or more specifically in certain embodiments, arteriography, of the area to be embolized may be first performed by injecting radiopaque contrast through a catheter inserted into an artery or vein (depending on the site to be embolized or injected) as an X-ray is taken. The catheter may be inserted either percutaneously or by surgery. The blood vessel may be then embolized by refluxing pharmaceutical compositions of the present invention through the catheter, until flow is observed to cease. Occlusion may be confirmed by repeating the angiogram. In embodiments where direct injection is used, the blood vessel is then infused with a pharmaceutical composition of the invention in the desired dose.

Embolization therapy generally results in the distribution of compositions containing inhibitors throughout the interstices of the tumor or vascular mass to be treated. The physical bulk of the embolic particles clogging the arterial lumen results in the occlusion of the blood supply. In addition to this effect, the presence of an anti-angiogenic factor(s) prevents the formation of new blood vessels to supply the tumor or vascular mass, enhancing the devitalizing effect of cutting off the blood supply. Direct intrarterial or intravenous generally results in distribution of compositions containing inhibitors throughout the interstices of the tumor or vascular mass to be treated as well. However, the blood supply is not generally expected to become occluded with this method.

In one aspect of the present invention, primary and secondary tumors of the liver or other tissues may be treated utilizing embolization or direct intraarterial or intravenous injection therapy. Briefly, a catheter is inserted via the femoral or brachial artery and advanced into the hepatic artery by steering it through the arterial system under fluoroscopic guidance. The catheter is advanced into the hepatic arterial tree as far as necessary to allow complete blockage of the blood vessels supplying the tumor(s), while sparing as many of the arterial branches supplying normal structures as possible. Ideally this will be a segmental branch of the hepatic artery, but it could be that the entire hepatic artery distal to the origin of the gastroduodenal artery, or even multiple separate arteries, will need to be blocked depending on the extent of tumor and its individual blood supply. Once the desired catheter position is achieved, the artery is embolized by injecting compositions (as described above) through the arterial catheter until flow in the artery to be blocked ceases, preferably even after observation for 5 minutes. Occlusion of the artery may be confirmed by injecting radio-opaque contrast through the catheter and demonstrating by fluoroscopy or X-ray film that the vessel which previously filled with contrast no longer does so. In embodiments where direct injection is used, the artery is infused by injecting compositions (as described above) through the arterial catheter in a desired dose. The same procedure may be repeated with each feeding artery to be occluded.

In most embodiments, the combination therapy will incorporate the substance or substances to be delivered in an amount sufficient to deliver to a patient a therapeutically effective amount of an incorporated therapeutic agent or other material as part of a prophylactic or therapeutic treatment. The desired concentration of active compound in the particle will depend on absorption, inactivation, and excretion rates of the drug as well as the delivery rate of the compound. It is to be noted that dosage values may also vary with the severity of the condition to be alleviated. It is to be further understood that for any particular subject, specific dosage regimens should be adjusted over time according to the individual need and the professional judgment of the person administering or supervising the administration of the compositions. Typically, dosing will be determined using techniques known to one skilled in the art. The selected dosage level will depend upon a variety of factors including the activity of the particular compound of the present invention employed, or the ester, salt or amide thereof, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

Dosage may be based on the amount of the composition per kg body weight of the patient. Other amounts will be known to those of skill in the art and readily determined. Alternatively, the dosage of the subject invention may be determined by reference to the plasma concentrations of the composition. For example, the maximum plasma concentration (Cmax) and the area under the plasma concentration-time curve from time 0 to infinity (AUC (0-4)) may be used. Dosages for the present invention include those that produce the above values for Cmax and AUC (0-4) and other dosages resulting in larger or smaller values for those parameters.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a combination therapy of an anti-LT-β-R binding molecule and at least one additional agent will be that amount of the combination therapy which is the lowest dose effective to produce a therapeutic effect. Such an effective dose will generally depend upon the factors described above.

In one embodiment, the effective dose of each agent in the combination therapy of the invention is the dose shown to be effective for that agent alone. In one embodiment, the effective dose of the anti-LT-β-R binding molecule is about 16 mg/m². In another embodiment, the effective dose of the anti-LT-β-R binding molecule is about 20 mg/m². In one embodiment, the effective dose of the agent that inhibits angiogenesis, e.g., an anti-VEGF antibody, is about 0.25-8 mg/kg, preferably about 4 mg/kg. (about 0.75-24 mg/m²). It will be understood by one of ordinary skill in the art that doses found to be effective in mouse models can easily be converted to doses appropriate for use in human subjects using a mathematical conversion, e.g., dose in mice in mg/kg can be divided by 12.1 and then multiplied by 37 to give the dose in mg/m² appropriate for humans.

In another embodiment, the effective dose of one or both agents in the combination therapy is a lower dose than that shown to be effective for each agent alone.

The precise time of administration and amount of any particular compound that will yield the most effective treatment in a given patient will depend upon the activity, pharmacokinetics, and bioavailability of a particular compound, physiological condition of the patient (including age, sex, disease type and stage, general physical condition, responsiveness to a given dosage and type of medication), route of administration, and the like. The guidelines presented herein may be used to optimize the treatment, e.g., determining the optimum time and/or amount of administration, which will require no more than routine experimentation consisting of monitoring the subject and adjusting the dosage and/or timing.

While the subject is being treated, the health of the patient may be monitored by measuring one or more of the relevant indices at predetermined times during a 24-hour period. Treatment, including supplement, amounts, times of administration and formulation, may be optimized according to the results of such monitoring. The patient may be periodically reevaluated to determine the extent of improvement by measuring the same parameters, the first such reevaluation typically occurring at the end of four weeks from the onset of therapy, and subsequent reevaluations occurring every four to eight weeks during therapy and then every three months thereafter. Therapy may continue for several months or even years, with a minimum of one month being a typical length of therapy for humans. Adjustments to the amount(s) of agent administered and possibly to the time of administration may be made based on these reevaluations.

Treatment may be initiated with smaller dosages which are less than the optimum dose of the compound. Thereafter, the dosage may be increased by small increments until the optimum therapeutic effect is attained.

Knowing this helps oncologists decide which drugs are likely to work well together and, if more than one drug will be used, plan exactly when each of the drugs should be given (in which order and how often).

In one embodiment of the invention, chemotherapeutic agents are further used in the combination treatment of the invention. Examples of chemotherapeutic agents which may be used include, but are not limited to the following: platinums (i.e., cis platinum), anthracyclines, nucleoside analogs (purine and pyrimidine), taxanes, camptothecins, epipodophyllotoxins, DNA alkylating agents, folate antagonists, vinca alkaloids, ribonucleotide reductase inhibitors, estrogen inhibitors, progesterone inhibitors, androgen inhibitors, aromatase inhibitors, interferons, interleukins, monoclonal antibodies, taxol, camptosar, adriamycin (dox), 5-FU and gemcitabine. Such chemotherapeutic agents may be employed in the practice of the invention by coadministration of the combination therapy and the chemotherapeutic. In one embodiment, an anti-LT-βR binding molecule is administered in combination with at least one additional agent and a chemotherapeutic agent selected from the group consisting of gemcitabine, adriamycin, Camptosar, carboplatin, cisplatin, and Taxol. Methods for treating cancer comprising administering an anti-lymphotoxin-beta receptor (LT-β-R) binding molecule and at least one chemotherapeutic agent are also described in U.S. Appln. 11/156,109, incorporated by reference herein.

In one embodiment, an anti-LT-βR binding molecule or a biologic agent is conjugated to a chemotherapeutic agent. In one embodiment, an anti-LT-β-R binding molecule or a biologic agent is nonconjugated to a chemotherapeutic agent. In another embodiment of the invention, the both biologic agent and an anti-LT-βR binding molecule are conjugated.

The combined use of an anti-LT-β-R binding molecule and at least one second agent as described herein (optionally in combination with other chemotherapeutics and/or biologic agents), may reduce the required dosage for any individual component, e.g., if the onset and duration of effect of the different components may be complimentary. In such combined therapy, the different active agents may be delivered together or separately, and simultaneously or at different times within the day. Toxicity and therapeutic efficacy of subject compounds may be determined by standard pharmaceutical procedures in cell cultures or experimental animals, e.g., for determining the LD50 and the ED50. Compositions that exhibit large therapeutic indices are preferred. Although compounds that exhibit toxic side effects may be used, care should be taken to design a delivery system that targets the compounds to the desired site in order to reduce side effects.

The data obtained from the cell culture assays and animal studies may be used in formulating a range of dosage for use in humans. The dosage of any supplement, or alternatively of any components therein, lies preferably within a range of circulating concentrations that include the ED50 with little or no toxicity. The dosage may vary within this range depending upon the dosage form employed and the route of administration utilized. For agents of the present invention, the therapeutically effective dose may be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information may be used to more accurately determine useful doses in humans. Levels in plasma may be measured, for example, by high performance liquid chromatography.

In the methods of the invention in which the at least one agent that inhibits angiogenesis is an antisense nucleic acid molecule, administration to a subject or generation of is typically in situ such that the antisense nucleic acid molecules hybridize with or bind to cellular mRNA and/or genomic DNA thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention include direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors known to one of skill in the art. To achieve sufficient intracellular concentrations of the antisense molecules, vector constructs in which the antisense nucleic acid molecule is placed under the control of a strong pol II or pol III promoter are preferred.

The administration of a nucleic acid molecule to a subject can be practiced either in vitro or in vivo (the latter is discussed further in the following subsection). For practicing the method in vitro, cells can be obtained from a subject by standard methods and incubated (i.e., cultured) in vitro with a nucleic acid molecule and subsequently administered to the subject. Methods for isolating immune cells are known in the art. For further discussion of ex vivo genetic modification of cells followed by readministration to a subject, see also U.S. Pat. No. 5,399,346 by W. F. Anderson et al.

In other embodiments, a nucleic acid molecule is administered to a subject in vivo, such as directly to an articulation site of a subject. For example, nucleic acids (e.g., recombinant expression vectors or antisense RNA) can be introduced into cells of a subject using methods known in the art for introducing nucleic acid (e.g., DNA) into cells in vivo. Examples of such methods include:

Direct Injection Naked DNA can be introduced into cells in vivo by directly injecting the DNA into the cells (see e.g., Acsadi et al. (1991) Nature 332:815-818; Wolff et al. (1990) Science 247:1465-1468). For example, a delivery apparatus (e.g., a “gene gun”) for injecting DNA into cells in vivo can be used. Such an apparatus is commercially available (e.g., from BioRad).

Receptor-Mediated DNA Uptake: Naked DNA can also be introduced into cells in vivo by complexing the DNA to a cation, such as polylysine, which is coupled to a ligand for a cell-surface receptor (see for example Wu, G. and Wu, C. H. (1988) J. Biol. Chem. 263:14621; Wilson et al. (1992) J. Biol. Chem. 267:963-967; and U.S. Pat. No. 5,166,320). Binding of the DNA-ligand complex to the receptor facilitates uptake of the DNA by receptor-mediated endocytosis. A DNA-ligand complex linked to adenovirus capsids which naturally disrupt endosomes, thereby releasing material into the cytoplasm can be used to avoid degradation of the complex by intracellular lysosomes (see for example Curiel et al. (1991) Proc. Natl. Acad. Sci. USA 88:8850; Cristiano et al. (1993) Proc. Natl. Acad. Sci. USA 90:2122-2126).

Retroviruses: Defective retroviruses are well characterized for use in gene transfer for gene therapy purposes (for a review see Miller, A. D. (1990) Blood 76:271). A recombinant retrovirus can be constructed having a nucleotide sequences of interest incorporated into the retroviral genome. Additionally, portions of the retroviral genome can be removed to render the retrovirus replication defective. The replication defective retrovirus is then packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which are well known to those skilled in the art. Examples of suitable packaging virus lines include ψ Crip, ψCre, ψ2 and ψAm. Retroviruses have been used to introduce a variety of genes into many different cell types, including epithelial cells, endothelial cells, lymphocytes, myoblasts, hepatocytes, bone marrow cells, in vitro and/or in vivo (see for example Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573). Retroviral vectors require target cell division in order for the retroviral genome (and foreign nucleic acid inserted into it) to be integrated into the host genome to stably introduce nucleic acid into the cell. Thus, it may be necessary to stimulate replication of the target cell.

Adenoviruses: The genome of an adenovirus can be manipulated such that it encodes and expresses a gene product of interest but is inactivated in terms of its ability to replicate in a normal lytic viral life cycle. See for example Berkner et al. (1988) BioTechniques 6:616; Rosenfeld et al. (1991) Science 252:431-434; and Rosenfeld et al. (1992) Cell 68:143-155. Suitable adenoviral vectors derived from the adenovirus strain Ad type 5 dl324 or other strains of adenovirus (e.g., Ad2, Ad3, Ad7 etc.) are well known to those skilled in the art. Recombinant adenoviruses are advantageous in that they do not require dividing cells to be effective gene delivery vehicles and can be used to infect a wide variety of cell types, including airway epithelium (Rosenfeld et al. (1992) cited supra), endothelial cells (Lemarchand et al. (1992) Proc. Natl. Acad. Sci. USA 89:6482-6486), hepatocytes (Herz and Gerard (1993) Proc. Natl. Acad. Sci. USA 90:2812-2816) and muscle cells (Quantin et al. (1992) Proc. Natl. Acad. Sci. USA 89:2581-2584). Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al. cited supra; Haj-Ahmand and Graham (1986) J. Virol. 57:267). Most replication-defective adenoviral vectors currently in use are deleted for all or parts of the viral E1 and E3 genes but retain as much as 80% of the adenoviral genetic material.

Adeno-Associated Viruses: Adeno-associated virus (AAV) is a naturally occurring defective virus that requires another virus, such as an adenovirus or a herpes virus, as a helper virus for efficient replication and a productive life cycle. (For a review see Muzyczka et al. Curr. Topics in Micro. and Immunol. (1992) 158:97-129). It is also one of the few viruses that may integrate its DNA into non-dividing cells, and exhibits a high frequency of stable integration (see for example Flotte et al. (1992) Am. J. Respir. Cell. Mol. Biol. 7:349-356; Samulski et al. (1989) J. Virol. 63:3822-3828; and McLaughlin et al. (1989) J. Virol. 62:1963-1973). Vectors containing as little as 300 base pairs of AAV can be packaged and can integrate. Space for exogenous DNA is limited to about 4.5 kb. An AAV vector such as that described in Tratschin et al. (1985) Mol. Cell. Biol. 5:3251-3260 can be used to introduce DNA into cells. A variety of nucleic acids have been introduced into different cell types using AAV vectors (see for example Hermonat et al. (1984) Proc. Natl. Acad. Sci. USA 81:6466-6470; Tratschin et al. (1985) Mol. Cell. Biol. 4:2072-2081; Wondisford et al. (1988) Mol. Endocrinol. 2:32-39; Tratschin et al. (1984) J. Virol. 51:611-619; and Flotte et al. (1993) J. Biol. Chem. 268:3781-3790).

The efficacy of a particular expression vector system and method of introducing nucleic acid into a cell can be assessed by standard approaches routinely used in the art. For example, DNA introduced into a cell can be detected by a filter hybridization technique (e.g., Southern blotting) and RNA produced by transcription of introduced DNA can be detected, for example, by Northern blotting, RNase protection or reverse transcriptase-polymerase chain reaction (RT-PCR). The gene product can be detected by an appropriate assay, for example by immunological detection of a produced protein, such as with a specific antibody, or by a functional assay to detect a functional activity of the gene product, such as an enzymatic assay.

### 5. Articles of Manufacture

The present invention provides kits and articles of manufacture for use of the methods of the present invention. The invention also pertains to packaged pharmaceutical compositions or kits for administering the anti-LT-β-R binding molecule used in the invention for the treatment of cancer. In one embodiment of the invention, the kit or article of manufacture, comprises an anti-LT-β-R binding molecule, and instructions for administration for treatment of cancer in combination with at least one additional agent, e.g., an agent that inhibits angiogenesis, e.g., a biologic agent. In another embodiment, the kit comprises a second container comprising at least one additional agent for use in a combination therapy with the anti-LT-β-R binding molecule. The instructions may describe how, e.g., intravenously, and when, e.g., at week 0 and week 2, the different doses of anti-LT-β-R binding molecule and the at least one additional agent shall be administered to a subject for treatment. In a further embodiment, the kit comprises a chemotherapeutic agent and/or instructions for administering a chemotherapeutic agent.

The package or kit alternatively can contain the anti-LT-β-R binding molecule and it can be promoted for use, either within the package or through accompanying information, for the uses or treatment of the disorders described herein. The packaged pharmaceuticals or kits further can include a second agent (as described herein, such as an agent that inhibits angiogenesis, e.g., a biologic agent) packaged with or co-promoted with instructions for using the second agent, e.g., an agent that inhibits angiogenesis, e.g., a biologic agent, with a first agent, e.g. an anti-LT-β-R binding molecule.

For example, an article of manufacture may comprise a packaging material, one or more anti-LT-β-R binding molecules and at least one additional agent as described above and optionally a label or package insert. In still other embodiments, the invention provides articles of manufacture comprising one or more anti-LT-β-R binding molecules and at least one additional agent and one or more devices for accomplishing administration of such compositions. For example, a kit may comprise a pharmaceutical composition comprising an anti-LT-β-R binding molecule and catheter for accomplishing direct intraarterial injection of the composition into a solid tumor. The articles of manufacture optionally include accessory components such as a second container comprising a pharmaceutically-acceptable buffer and instructions for using the composition.

### EXAMPLES

The present invention is further illustrated by the following examples which should not be construed as limiting in any way.

### Materials and Methods

### WiDr Mouse Model

In order to study the effects of biologic agents in combination with huCBE11, the WiDr xenograft model was used. CBE11 has been shown to exhibit antitumor activity against WiDr tumors grown as xenografts in mice with severe combined immunodeficiency (SCID) (Browning et al. (1996) J. Exp. Med. 183:867). Therapeutic agents, i.e. LTβR antibody and biologic agents, were administered to athymic nude mice who had been implanted with WiDr tumor cells. Antitumor activity, was studied according to the growth of WiDr xenograft human colorectal tumors, wherein treatment was initiated on an established, preformed tumor mass.

WiDr cells were obtained from the American Type Culture Collection (Manassas, Va.). Cells were grown in vitro in 90% Eagle's Minimum Essential Medium with 2 mM L-glutamine and Earle's Balanced Salt Solution (BSS) adjusted to contain 1.5 g/L sodium bicarbonate, 0.1 mM non-essential amino acids, and 1 mM sodium pyruvate plus 10% fetal bovine serum (FBS) without antibiotics (5% CO₂). Bacterial cultures were performed on aliquots of the tumor homogenate preparation that was implanted into the mice to ensure that all cultures were negative for bacterial contamination at both 24 and 48 hours post implant.

An inoculum of 2×10⁶ WiDr cells in 200 μL RPMI 1640 without serum was implanted subcutaneously into the right flank area on Day 0. Tumor weight and body weight measurements were recorded twice-weekly beginning on Day 3. When the tumors measured approximately 5 mm in length by 5 mm in width, mice were randomized to treatment and control groups. Body weight measurements were recorded twice-weekly beginning on Day 0.

### KM-20L2 Mouse Model

In order to study the effects of biologic agents in combination with huCBE11, the KM-20L2 xenograft model was used. Therapeutic agents, i.e. LTβR antibody and biologic agents, were administered to athymic nude mice who had been implanted with KM-20L2 tumor cells. Antitumor activity was studied according to the growth of KM-20L2 xenograft, wherein treatment was initiated on an established, preformed tumor mass.

KM-20L2 were obtained from the from the NCI tumor repository. Cells were grown in 90% RPMI-1640 with 10% fetal bovine serum without antibiotics. Bacterial cultures were performed on aliquots of the tumor cell homogenate preparation that were implanted into the mice to ensure that all cultures were negative for bacterial contamination at both 24 and 48 hours post implant.

An inoculum of 2×10⁶ or 3×10⁶ KM-20L2 cells in medium without serum was implanted subcutaneously into the right flank area of the mouse on Day 0. Tumor size measurements were recorded regularly. When the tumors measured approximately 5 mm in length by 5 mm in width (65 mg), mice were randomized into treatment and control groups.

### Tumor Measurements

Tumor measurements were determined using Vernier calipers. Tumor size measurements were recorded regularly according to the study, until the termination of the study. The formula to calculate volume for a prolate ellipsoid was used to estimate tumor volume (mm³) from 2-dimensional tumor measurements: tumor volume (mm³)=(length×width2 [L×W²])÷2. Assuming unit density, tumor volume is converted to tumor weight (i.e., 1 mm³=1 mg). Tumor growth inhibition was assessed as % T/C, where T is the mean tumor weight of the treatment group and C is the mean tumor weight of the control group. A % T/C value of 42% or less for this type of study is considered indicative of meaningful activity by the National Cancer Institute (USA). Animals were sacrificed accordingly.

### Statistical Analysis

Statistical analysis of the tumor weight measurements was performed according to standard statistical methods. Mean, standard deviation (SD), and standard error of the mean (SEM) were determined for body weight and tumor weight for all dose groups at all assessments. Student's t test was performed on mean tumor weights at each assessment, including at the end of each study, to determine whether there were any statistically significant differences between each treatment group and the vehicle control group and between each combination treatment group and the respective huCBE11 group.

Treatment efficacy was determined by comparing each treatment group's tumor weight with the control group's tumor weight. Further statistical analysis was performed accordingly.

### Example 1

### Reduction of Tumor Size Using an LTβR Antibody in Combination with Biologic Agent

A. Reduction of Tumor Size Using a Combination of huCBE11 and Bevacizumab in the KM-20L2 Human Colon Adenocarcinoma Xenograft Model

In order to determine whether administration of a biologic agent, e.g., a biologic that inhibits angiogenesis, e.g., an anti-VEGF antibody, e.g., bevacizumab (Avastin), in combination with huCBE11 is more effective at reducing tumor size than each compound alone, bevacizumab was administered in combination with huCBE11 using the KM-20L2 (human colon adenocarcinoma) xenograft model.

A dosing range study was performed to determine the appropriate bevacizumab and huCBE11 dose(s) for studying the antitumor effects of bevacizumab and huCBE11. The dosing study also examined the antitumor efficacy of each agent at inhibiting tumor growth individually. Athymic nude mice bearing approximately 65 mg KM-20L2 tumors (approximately 6-7 days post implantation) were treated with either saline (control) (n=15; 200 μl intraperitoneally, twice per week) or huCBE11 (n=10 per dose; 0.2 mg/kg, 2 mg/kg, 4 mg/kg, or 20 mg/kg intraperitoneally, twice per week). Similarly, athymic nude mice bearing approximately 75 mg KM-20L2 tumors or 100 mg KM-20L2 tumors (approximately 6-7 days post implantation) were treated with either saline (control) (n=15; 200 μl intraperitoneally, twice per week) or bevacizumab (n=10 per dose; 1 mg/kg, 2 mg/kg, or 4 mg/kg intraperitoneally, twice per week). Tumor weight was measured on day 5 and regularly thereafter until sacrifice of the animals.

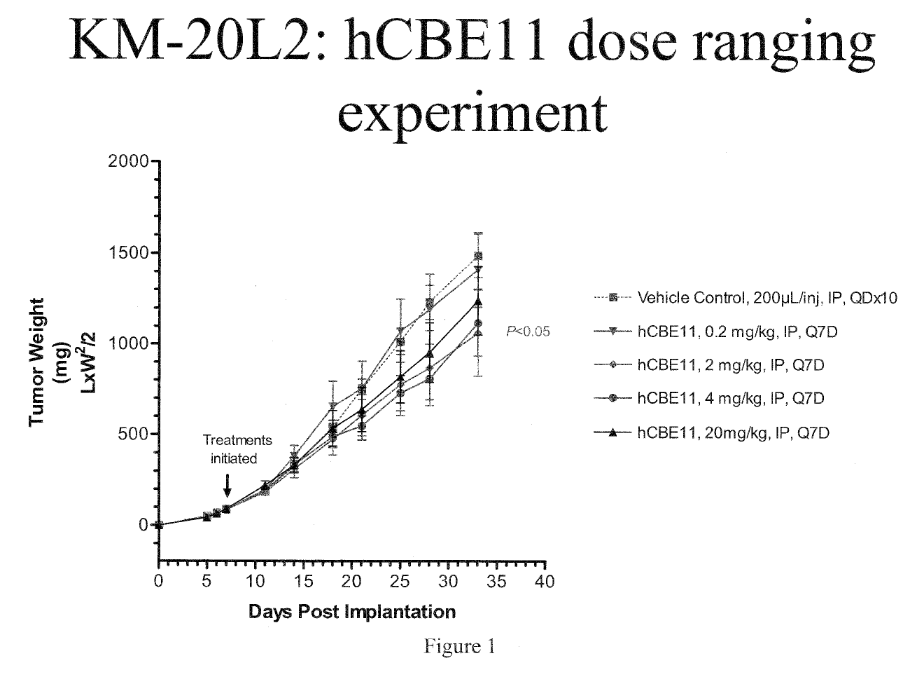

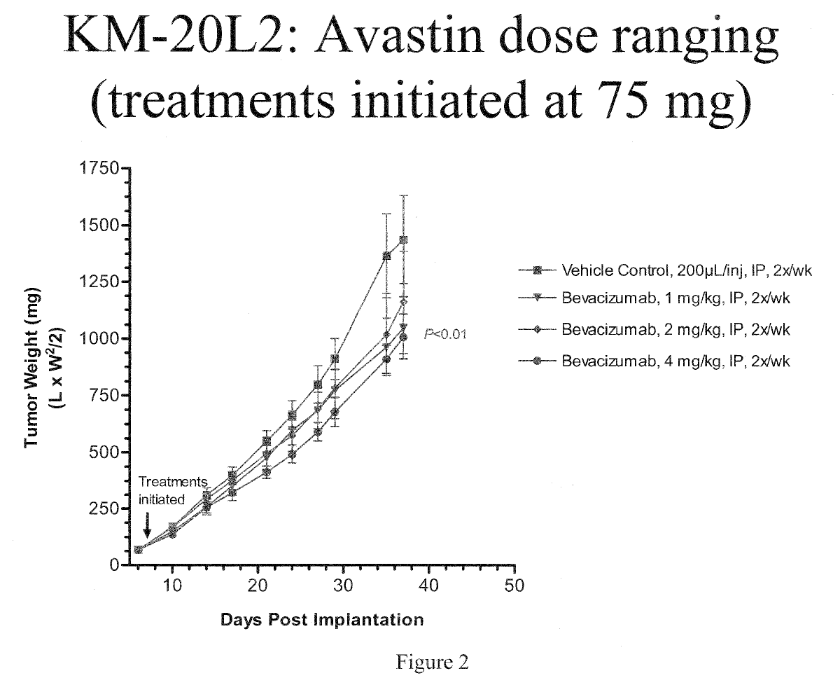

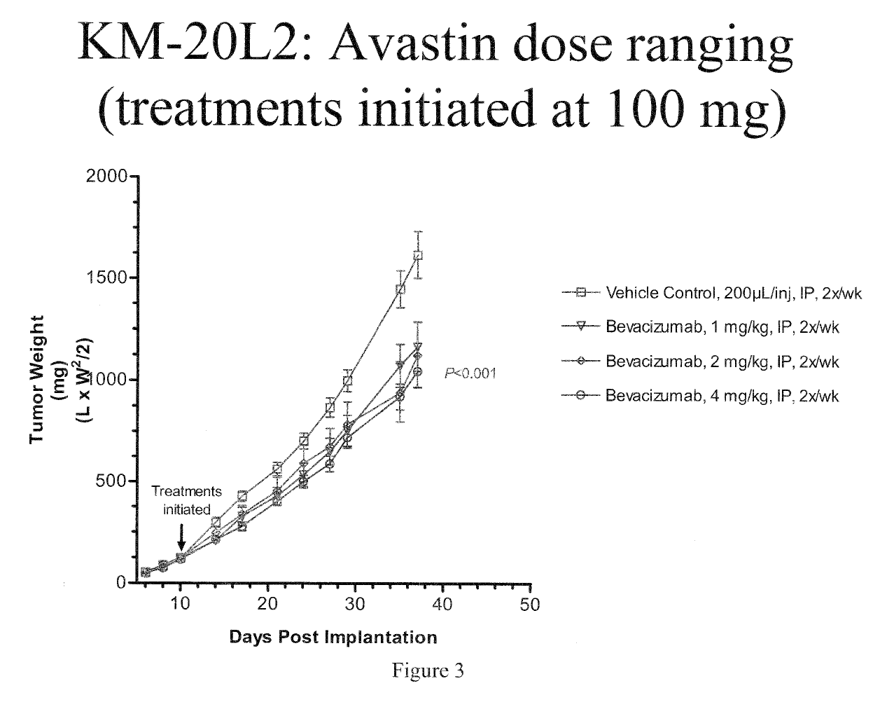

Tumor weight in the 0.2 mg/kg and 20 mg/kg huCBE11 dose groups did not differ significantly from the saline control group at day 35 post implant. However, huCBE11 produced a significant inhibition of KM-202L2 human colon adenocarcinoma tumor weight in nude mice at a dose of 2 mg/kg or 4 mg/kg (P<0.05) (FIG. 1). In parallel studies, it was determined that on day 38, bevacizumab produced a significant inhibition of tumor weight at a dose of 4 mg/kg for tumors weighing either approximately 75 mg at the initiation of treatment (P<0.01) (FIG. 2) or 100 mg/kg at the initiation of treatment (P<0.001) (FIG. 3).

In order to determine whether the combination treatment of bevacizumab and huCBE11 had a significant increase in inhibiting tumor weight, a combination study was performed on athymic nude mice bearing approximately 65 mg KM-20L2 tumors. This study compared the effect of huCBE11 (2 mg/kg) and bevacizumab (4 mg/kg) to determine efficacy.

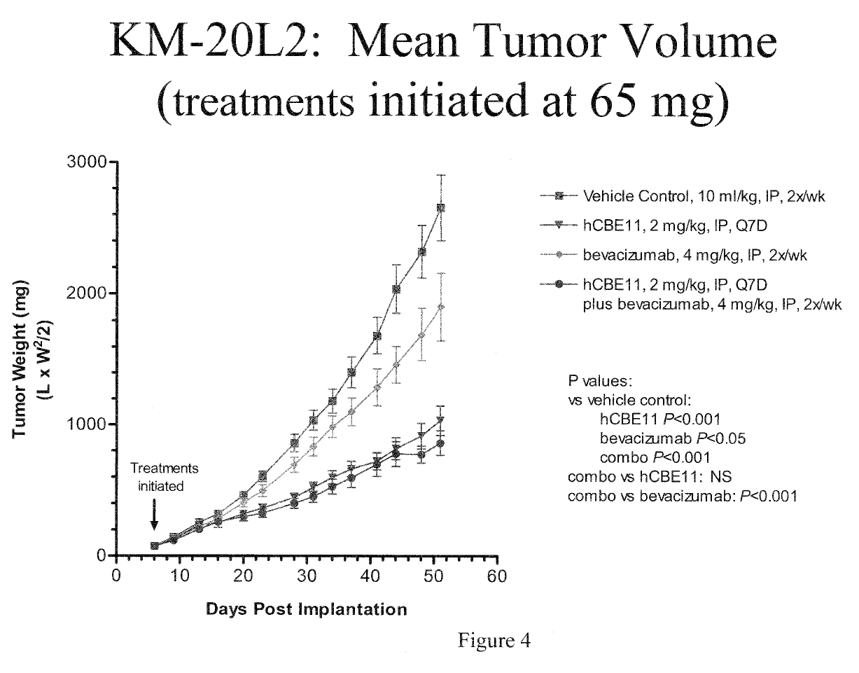

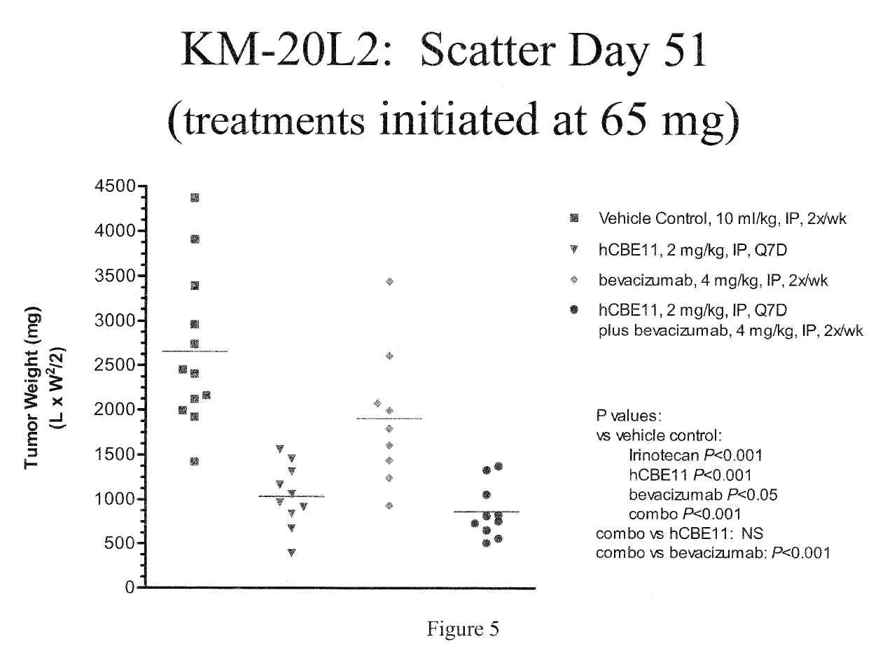

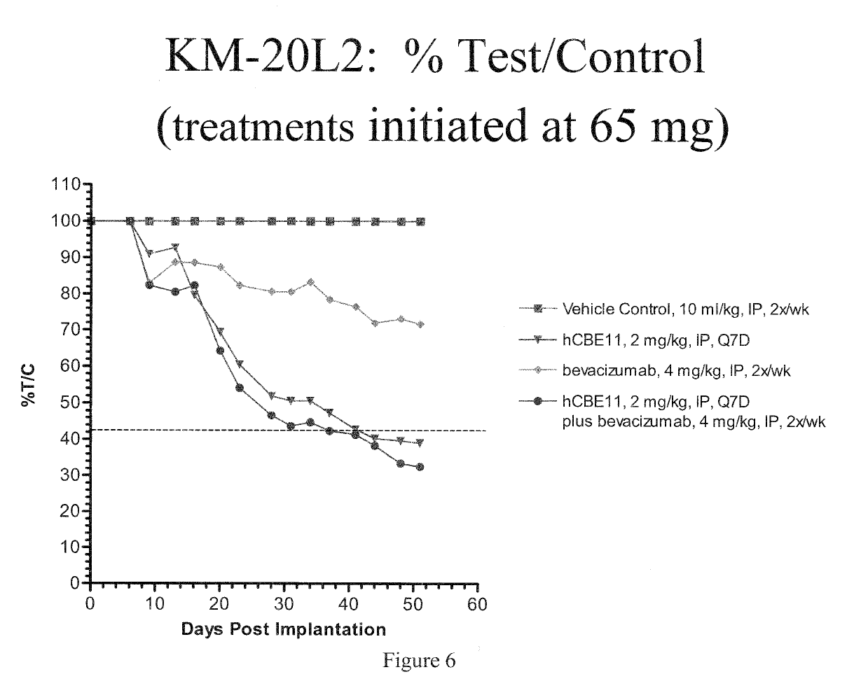

Results from the combination studies (shown in FIGS. 4-6) demonstrate that compared to vehicle or treatment with bevacizumab alone, huCBE11 in combination with bevacizumab significantly decreases tumor weight in treated mice bearing approximately 65 mg KM-20L2 tumors at the initiation of treatment (P=<0.001). However, compared to treatment with huCBE11 alone, huCBE11 in combination with bevacizumab does not significantly decreases tumor weight in treated mice bearing approximately 65 mg KM-20L2 tumors at the initiation of treatment.

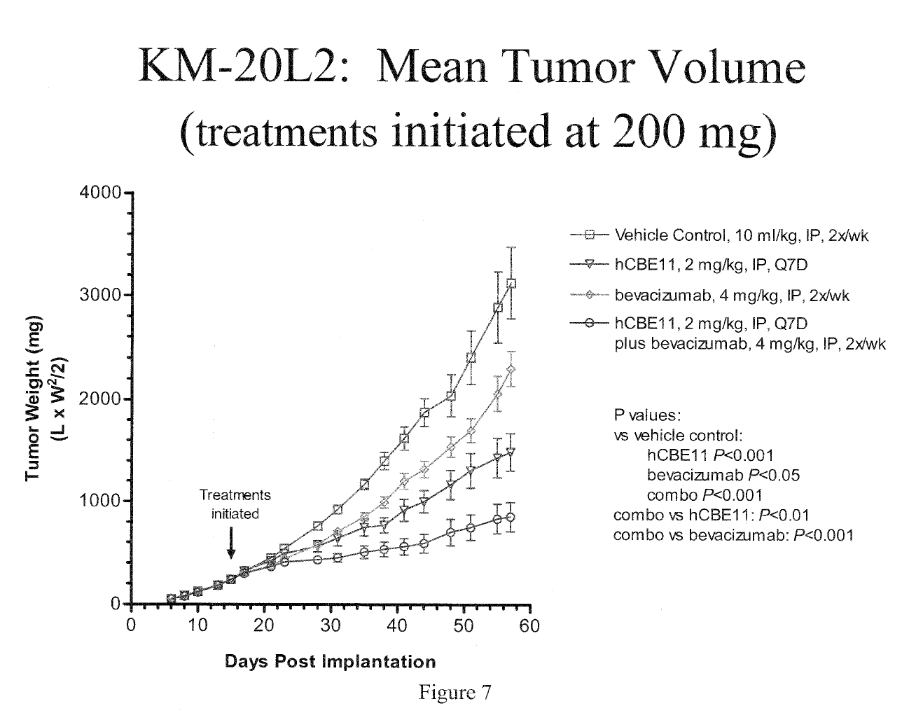

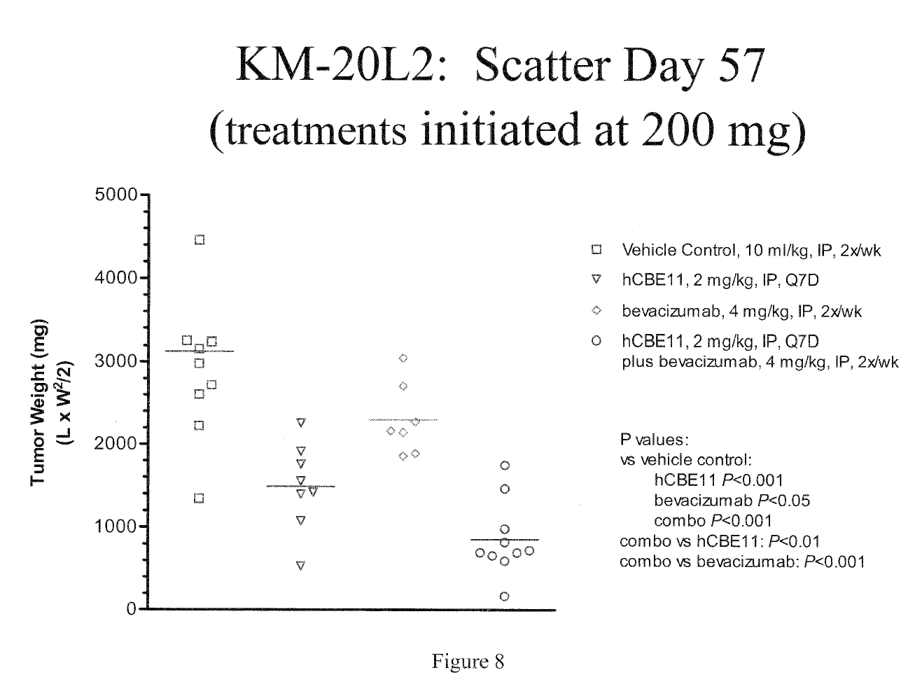

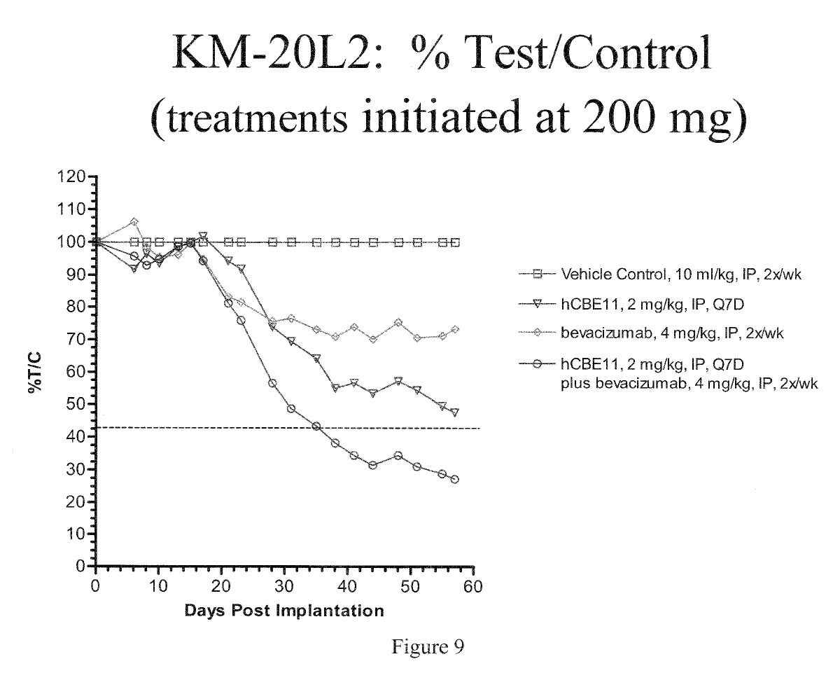

Surprisingly, compared to vehicle treatment or treatment with huCBE11 or bevacizumab alone, the combination of huCBE11 and bevacizumab significantly decreases tumor weight in treated mice bearing approximately 200 mg KM-20L2 tumors at the initiation of treatment (FIGS. 7 and 8). As shown in FIG. 9, the combination treatment of a 200 mg KM-20L2 tumor with huCBE11 and bevacizumab has a % T/C of 26% (and, thus, a % tumor inhibition of 74%), well below the significant 42% level and lower than the % T/C observed with the treatment of a large tumor with either huCBE11 and bevacizumab alone. This enhanced reduction in tumor size of a larger tumor was unexpected since previous analyses have demonstrated that bevacizumab is not effective at reducing the size of large tumors.

B. Reduction of Tumor Size Using a Combination of huCBE11 and Bevacizumab in the WiDr Human Colon Colorectal Xenograft Model

In order to determine whether administration of biologic, e.g., a biologic that inhibits angiogenesis, e.g., an anti-VEGF antibody, e.g., bevacizumab (Avastin), in combination with huCBE11 is effective in reducing tumor size, bevacizumab was administered in combination with huCBE11 using the WiDr (human colorectal) xenograft model.

A dosing range study was performed to determine the appropriate bevacizumab and huCBE11 dose(s) for studying the antitumor effects of bevacizumab and huCBE11. The dosing study also examined the antitumor efficacy of each agent at inhibiting tumor growth individually. Athymic nude mice bearing approximately 65 mg WiDr tumors (approximately 6-7 days post implantation) were treated with either saline (control) (n=15; 200 μl intraperitoneally, twice per week) or huCBE11 (n=10 per dose; 0.2 mg/kg, 2 mg/kg, 4 mg/kg, or 20 mg/kg intraperitoneally, twice per week). Similarly, athymic nude mice bearing approximately 100 mg WiDr tumors or 100 mg WiDr tumors (approximately 6-7 days post implantation) were treated with either saline (control) (n=15; 200 μl intraperitoneally, twice per week) or bevacizumab (n=10 per dose; 1 mg/kg, 2 mg/kg, or 4 mg/kg intraperitoneally, twice per week). Tumor weight was measured on day 5 and regularly thereafter until sacrifice of the animals.

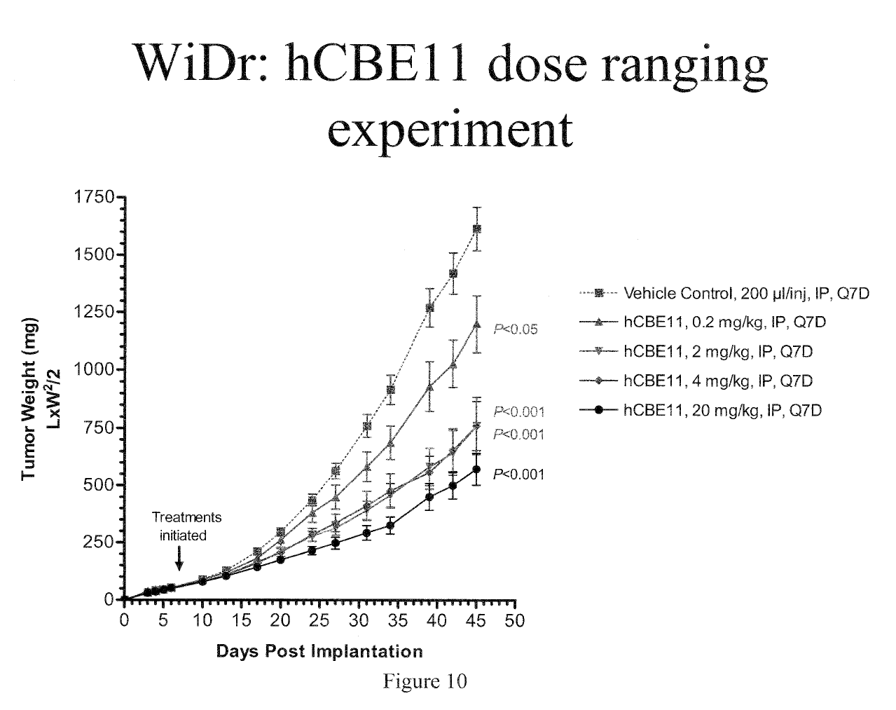

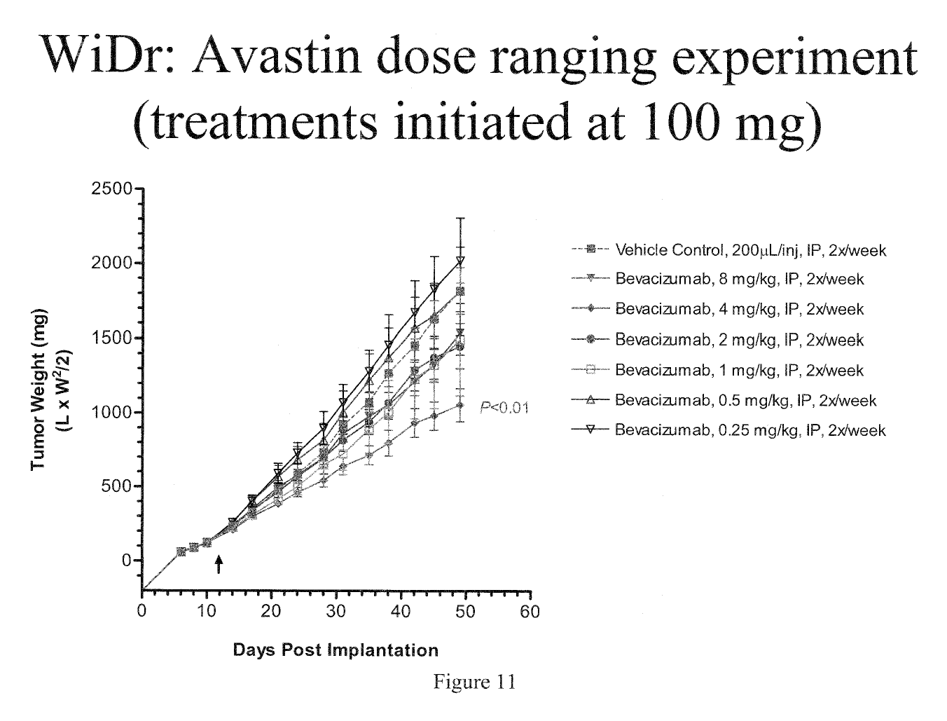

Tumor weight in the 0.2 mg/kg and 20 mg/kg huCBE11 dose groups did not differ significantly from the saline control group at day 35 post implant. However, huCBE11 produced a significant inhibition of WiDr human colon tumor weight in nude mice at a dose of 2 mg/kg or 4 mg/kg (P<0.05) (FIG. 10). In parallel studies, it was determined that on day 38, bevacizumab produced a significant inhibition of tumor weight at a dose of 4 mg/kg (P<0.01) for tumors weighing either approximately 100 mg/kg at the initiation of treatment (FIG. 11).

In order to determine whether the combination treatment of bevacizumab and huCBE11 had a significant increase in inhibiting tumor weight, a combination study was performed on athymic nude mice bearing approximately 65 mg WiDr tumors. This study compared the effect of huCBE11 (2 mg/kg) and bevacizumab (4 mg/kg) to determine efficacy.

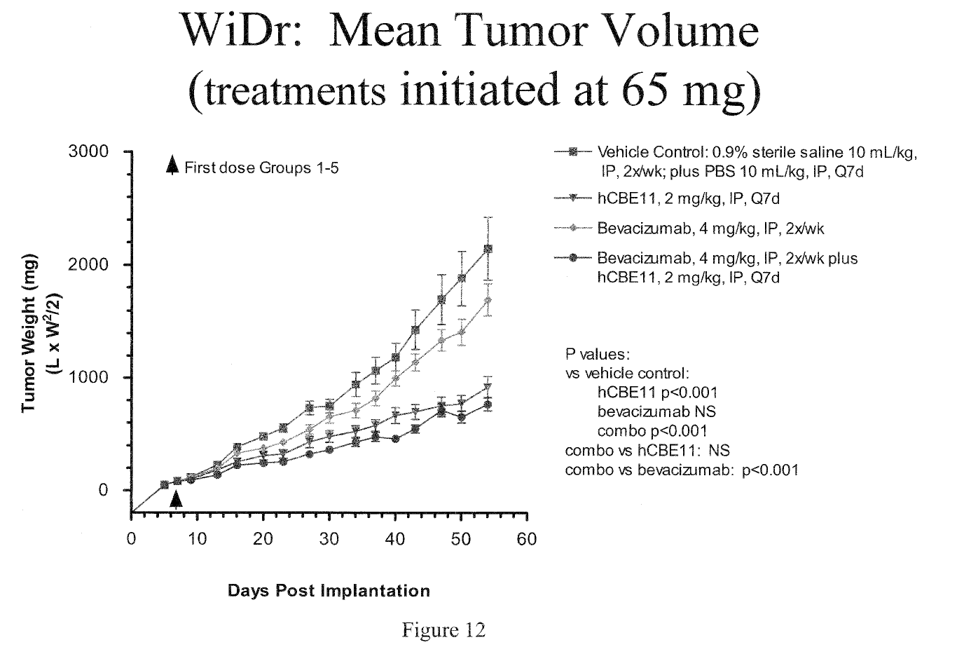

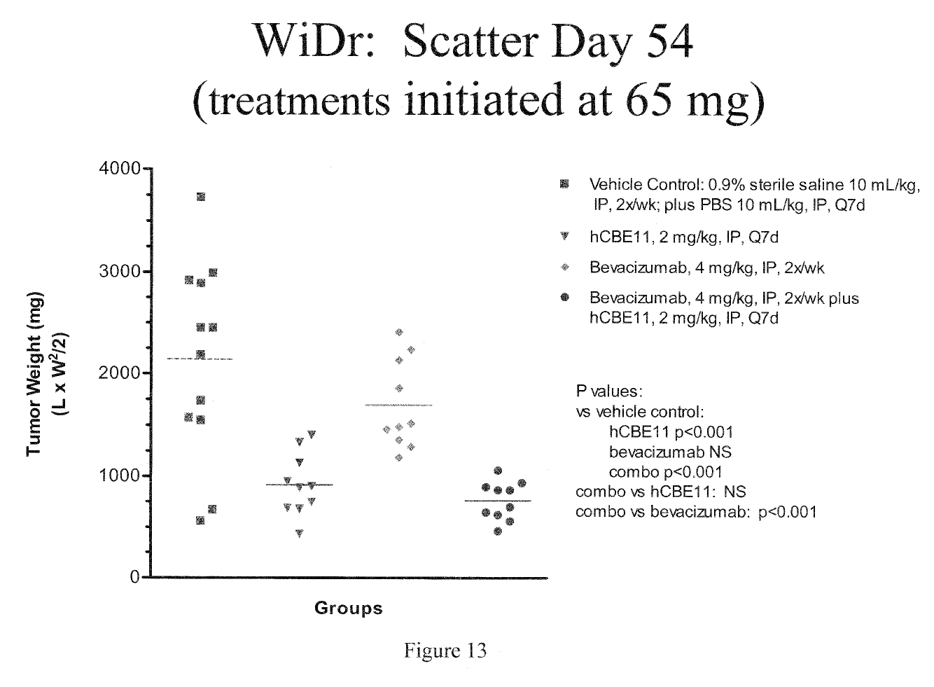

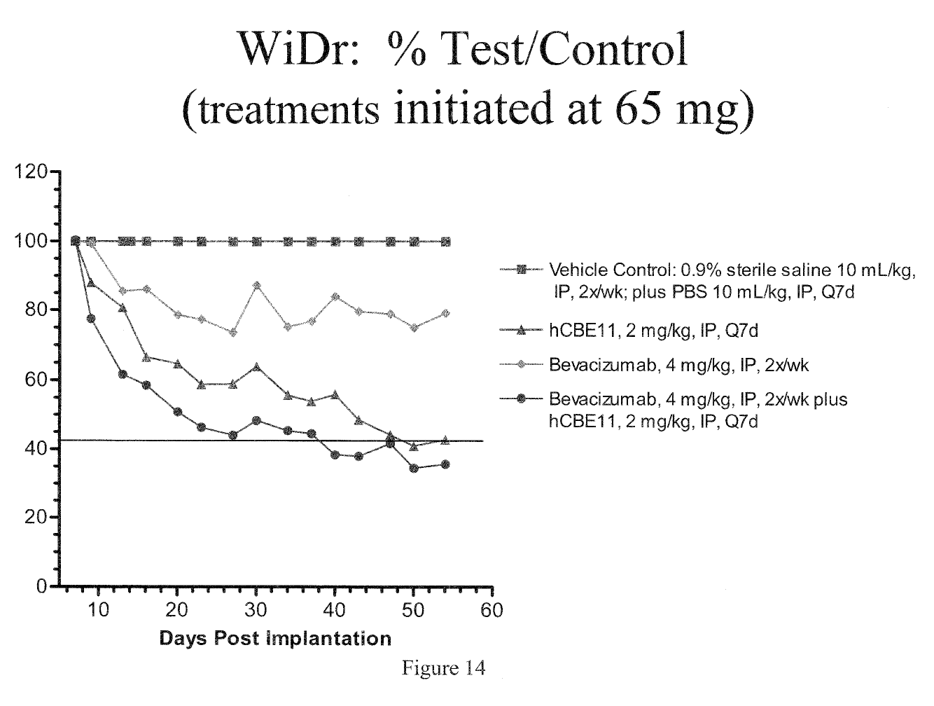

Results from the combination studies (shown in FIGS. 12-14) demonstrate that compared to vehicle or treatment with huCBE11 or bevacizumab alone, huCBE11 in combination with bevacizumab significantly decreases tumor weight in treated mice bearing approximately 65 mg WiDr tumors at the initiation of treatment. However, compared to treatment with huCBE11 alone, huCBE11 in combination with bevacizumab does not significantly decreases tumor weight in treated mice bearing approximately 65 mg WiDr tumors at the initiation of treatment.

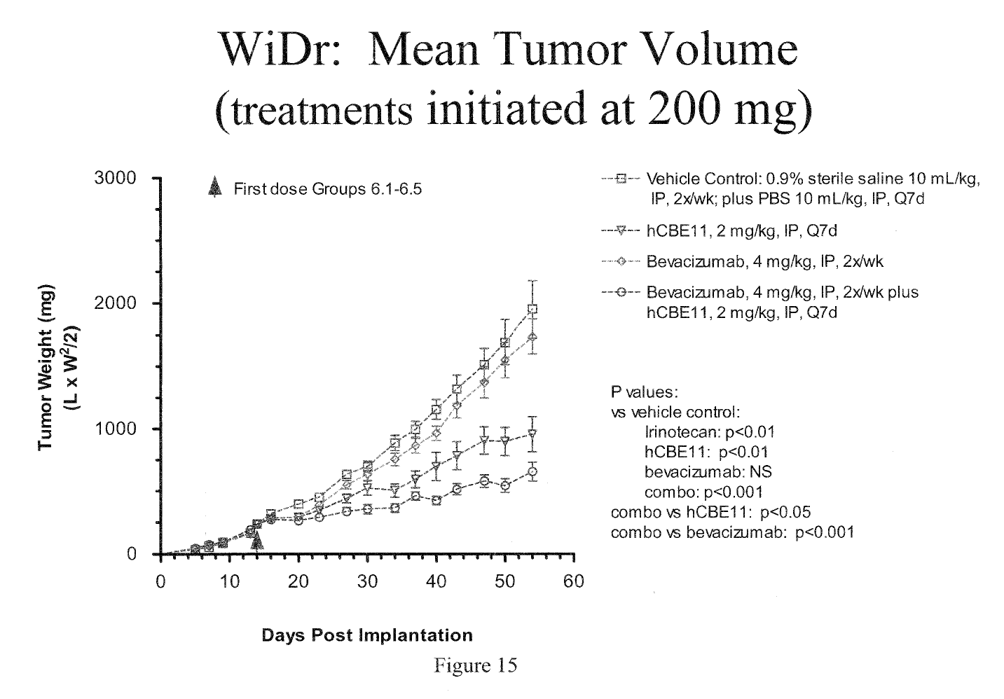

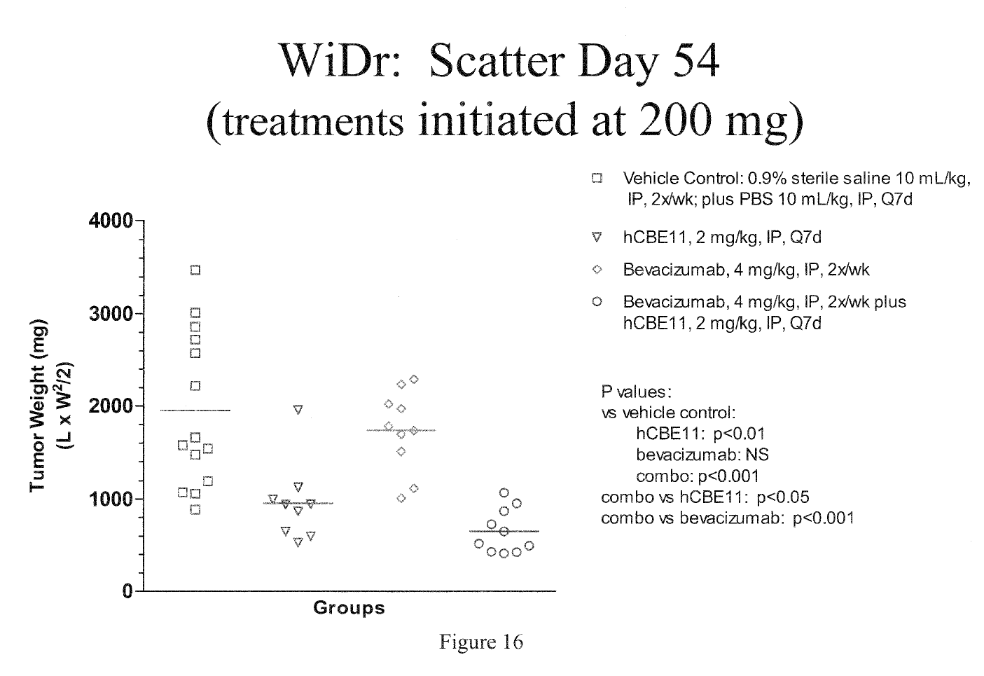

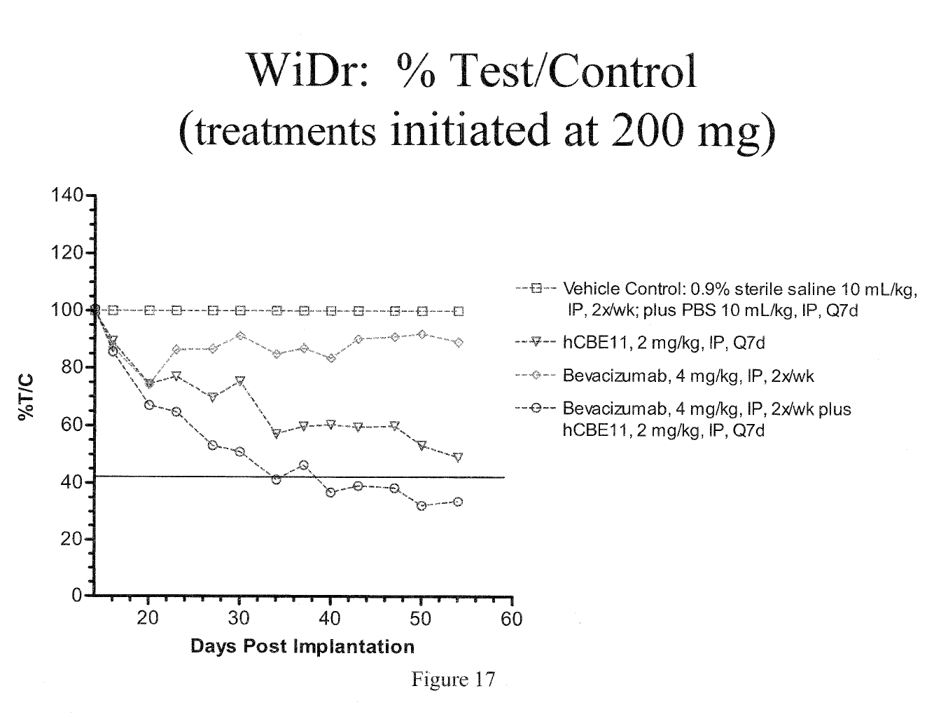

Similarly to the results obtained using the KM-20L2, compared to vehicle treatment or treatment with huCBE11 or bevacizumab alone, the combination of huCBE11 and bevacizumab significantly decreases tumor weight in treated mice bearing approximately 200 mg WiDr tumors at the initiation of treatment (FIGS. 15 and 16). As shown in FIG. 17, the combination treatment of a 200 mg WiDr tumor with huCBE11 and bevacizumab has a % T/C of 37% (and, thus, a % tumor inhibition of 63%), well below the significant 42% level and lower than the % T/C observed with the treatment of a large tumor with either huCBE11 or bevacizumab alone. This enhanced reduction in tumor size of a larger tumor was unexpected since previous analyses have demonstrated that bevacizumab is not effective at reducing the size of large tumors.

### EQUIVALENTS

The present invention provides among other things combination therapeutics involving LT-β-R antibodies. While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations.

All publications and patents mentioned herein, including those items listed below, are hereby incorporated by reference in their entirety as if each individual publication or patent was specifically and individually indicated to be incorporated by reference. In case of conflict, the present application, including any definitions herein, will control.

We claim:

1. A method for reducing tumor size in a subject having a tumor of a size greater than about 2 mm×2 mm, comprising administering an anti-lymphotoxin-beta receptor (LT-β-R) binding molecule, or an antigen-binding fragment thereof, and at least one additional agent to the subject, such that the tumor size is reduced.

2. A method for decreasing vascularization of a solid tumor in a subject having a solid tumor, comprising administering an anti-LT-β-R binding molecule, or an antigen-binding fragment thereof, and at least one additional agent to the subject, such that vascularization of the solid tumor is decreased.

3. A method for increasing permeability of a solid tumor in a subject having a solid tumor, comprising administering an anti-LT-β-R binding molecule, or an antigen-binding fragment thereof, and at least one additional agent to the subject, such that permeability of the solid tumor to the anti-LT-β-R binding molecule, or antigen-binding fragment thereof, is increased.

4. The method of claim 1, wherein the at least one additional agent is administered to the subject either prior to administration of the anti-LT-β-R binding molecule, or antigen-binding fragment thereof or concomitantly with the anti-LT-β-R binding molecule, or antigen-binding fragment thereof.

5. The method of claim 1, wherein the at least one additional agent inhibits angiogenesis.

6. The method of claim 2, wherein the at least one additional agent inhibits angiogenesis.

7. The method of claim 3, wherein the at least one additional agent inhibits angiogenesis.

8. The method of claim 5, wherein the agent that inhibits angiogenesis is selected from the group consisting of gefitinib, imatinib mesylate, erlotinib, and bortezomib.

9. The method of claim 6, wherein the agent that inhibits angiogenesis is selected from the group consisting of gefitinib, imatinib mesylate, erlotinib, and bortezomib.

10. The method of claim 7, wherein the agent that inhibits angiogenesis is selected from the group consisting of gefitinib, imatinib mesylate, erlotinib, and bortezomib.

11. The method of claim 5, wherein the agent that inhibits angiogenesis is a biologic agent.

12. The method of claim 6, wherein the agent that inhibits angiogenesis is a biologic agent.

13. The method of claim 7, wherein the agent that inhibits angiogenesis is a biologic agent.

14. The method of claim 11, wherein the biologic agent is an antibody, or antigen binding fragment thereof.

15. The method of claim 12, wherein the biologic agent is an antibody, or antigen binding fragment thereof.

16. The method of claim 13, wherein the biologic agent is an antibody, or antigen binding fragment thereof.

17. The method of claim 11, wherein the biologic agent that inhibits angiogenesis is an anti-VEGF antibody or an anti-EGFR antibody.

18. The method of claim 12, wherein the biologic agent that inhibits angiogenesis is an anti-VEGF antibody or an anti-EGFR antibody.

19. The method of claim 13, wherein the biologic agent that inhibits angiogenesis is an anti-VEGF antibody or an anti-EGFR antibody.

20. The method of claim 11, wherein the biologic agent is selected from the group consisting of: bevacizumab, cetuximab, rituximab, trastuzumab, tositumomab, ibritumomab, alelmtuzumab, epratuzumab, gemtuzumab ozogamicin, oblimersen, and panitumumab.

21. The method of claim 12, wherein the biologic agent is selected from the group consisting of: bevacizumab, cetuximab, rituximab, trastuzumab, tositumomab, ibritumomab, alelmtuzumab, epratuzumab, gemtuzumab ozogamicin, oblimersen, and panitumumab.

22. The method of claim 13, wherein the biologic agent is selected from the group consisting of: bevacizumab, cetuximab, rituximab, trastuzumab, tositumomab, ibritumomab, alelmtuzumab, epratuzumab, gemtuzumab ozogamicin, oblimersen, and panitumumab.

23. The method of claim 1, wherein the anti-LT-β-R binding molecule, or antigen-binding fragment thereof, comprises a humanized CBE11 (huCBE11), or an antigen binding fragment thereof.

24. The method of claim 2, wherein the anti-LT-β-R binding molecule, or antigen-binding fragment thereof, comprises a humanized CBE11 (huCBE11), or an antigen binding fragment thereof.

25. The method of claim 3, wherein the anti-LT-β-R binding molecule, or antigen-binding fragment thereof, comprises a humanized CBE11 (huCBE11), or an antigen binding fragment thereof.

26. The method of claim 1, wherein the tumor is selected from the group consisting of a colon tumor, a cervical tumor, a gastric tumor, a carcinoma, and a pancreatic tumor.

27. The method of claim 1, wherein the tumor is a size selected from the group consisting of: at least about 1 mm×1 mm, at least about 2 mm×2 mm, and a volume of at least about 1 cm³.

28. The method of claim 1, further comprising administering a chemotherapeutic agent to the subject.

29. The method of claim 28, wherein the chemotherapeutic agent is selected from the group consisting of gemcitabine, adriamycin, Camptosar, carboplatin, cisplatin, and Taxol.

30. The method of claim 5, wherein the administration of the anti-lymphotoxin-beta receptor (LT-β-R) binding molecule, or an antigen-binding fragment thereof, and at least one agent that inhibits angiogenesis results in a % tumor inhibition of about 58% or greater.

31. An article of manufacture comprising:
a) a packaging material;
b) an anti-LT-β-R binding molecule, or antigen-binding fragment thereof; and
c) a label or package insert contained within the packaging material indicating that the anti-LT-β-R binding molecule, or antigen-binding fragment thereof, can be administered with at least one additional agent that inhibits angiogenesis.

32. The article of claim 31, wherein the anti-LT-β-R binding molecule, or antigen binding fragment thereof comprises a huCBE11 antibody, or antigen-binding fragment thereof, and/or, wherein the additional agent is either bevacizumab or cetuximab.
